(19) United States
(12) Patent Application Publication
(10) Pub. No.: US 2021/0155660 A1
(43) Pub. Date: May 27, 2021

(54) RATIONALLY DESIGNED VIRUS-LIKE PARTICLES FOR MODULATION OF CHIMERIC ANTIGEN RECEPTOR (CAR)-T-CELL THERAPY

(71) Applicant: Ruprecht-Karls-Universität Heidelberg, Heidelberg (DE)

(72) Inventors: Patrick Schmidt, Heidelberg (DE); Silke Uhrig-Schmidt, Heidelberg (DE); Inka Zörnig, Heidelberg (DE); Krysten Doll, Heidelberg (DE); Oliver Müller, Heidelberg (DE); Dirk Jäger, Heidelberg (DE)

(21) Appl. No.: 16/641,611

(22) PCT Filed: Aug. 29, 2018
(86) PCT No.: PCT/EP2018/073276

§ 371 Date: Feb. 24, 2020

(30) Foreign Application Priority Data
Aug. 29, 2017 (EP) 17188336.6

(51) Int. Cl. C07K 14/075; C12N 15/86

(52) U.S. Cl. CPC C07K 14/075; C12N 15/86; A61K 38/00; C12N 2750/14143; C12N 2750/14123

(57) ABSTRACT

The present invention relates to a modified viral structural protein (VSP) as a tool for specifically targeting a chimeric antigen receptor (CAR) expressed on cells of the immune system. The modified VSPs can assemble into virus like particles (VLP). Exposed areas of the VSPs are modified to comprise in a region located atthe surface of a higher order structure, e.g. such as a capsomeric structure, a capsid, a VLP, a viral vector or a virus, a ligand specifically binding to a CAR (LCAR). The present invention thus, provides a modified VSP. The invention also relates to a nucleic acid encoding said VSP. Further, the invention relates to a capsomeric structure, a capsid, a VLP, a viral vector or a virus comprising at least one VSP. Further, the invention relates to a pharmaceutical composition comprising the VSP, the nucleic acid, the capsomeric structure, the capsid, the VLP, the viral vector or the virus comprising at least one VSP. Further, the invention relates to a VSP, a capsomeric structure, a capsid, a VLP, a viral vector or a virus for use in medicine, in particular for use in decreasing or limiting an immune response, treating or preventing tumor lysis syndrome or for treating an immune disease in a patient.

The present invention relates to a modified viral structural protein (VSP) as a tool for specifically targeting a chimeric antigen receptor (CAR) expressed on cells of the immune system. The modified VSPs can assemble into virus-like particles (VLP). Exposed areas of the VSPs are modified to comprise in a region located at the surface of a higher order structure, e.g. such as a capsomeric structure, a capsid, a VLP, a viral vector or a virus, a ligand specifically binding to a CAR (LCAR). The present invention thus, provides a modified VSP. The invention also relates to a nucleic acid encoding said VSP. Further, the invention relates to a capsomeric structure, a capsid, a VLP, a viral vector or a virus comprising at least one VSP. Further, the invention relates to a pharmaceutical composition comprising the VSP, the nucleic acid, the capsomeric structure, the capsid, the VLP, the viral vector or the virus comprising at least one VSP. Further, the invention relates to a modified VSP or a capsomeric structure, a capsid, a VLP, a viral vector or a virus comprising such modified VSP for use in medicine, in particular for use in preventing, decreasing or limiting an immune response, in particular for preventing, decreasing or treating tumor lysis syndrome, cytokine release syndrome, neurologic toxicity, “on target/off tumor” recognition, graft-versus-host disease (GVHD) and/or anaphylaxis in a patient.

### BACKGROUND OF THE INVENTION

In therapy of cancer approaches like chimeric antigen receptor (CAR)-T-cell therapy are promising but also go along with certain drawbacks, such as severe adverse reactions, including among others cytokine release syndrome, neurologic toxicity, “on target/off tumor” recognition, graft-versus-host disease (GVHD) and anaphylaxis. The CAR-T-cell therapy is a type of treatment in which a patient's T cells are modified in the laboratory so they will attack cancer cells. T cells are taken from a patient's blood. Then the gene encoding a special receptor that binds to a certain protein on the patient's cancer cells is introduced into the cell ex vivo. The special receptor is called a chimeric antigen receptor (CAR). Large numbers of CAR T cells are grown in the laboratory and administered to the patient by infusion. First clinical trials with CAR T cells focused on B cell leukemia or lymphoma. These diseases are characterized by the occurrence of tumor cells expressing (amongst others) the CD19 molecule. The CAR in these trials is specifically binding a motif of the CD19 protein, which is called a tumor specific epitope or LCAR. Establishment of a binding pair LCAR/CAR results in a specific activation of the T cell against the tumor cell and subsequent killing of the latter one. Activated T cells release a variety of signal molecules (cytokines) into the environment after recognition and killing of the target cell to attract other immunologically active cells. When large numbers of CAR T cells are infused into the patient they face, especially in late stage patients, a high number of target cells. A successful eradication of these tumor cells goes then hand in hand with a massive release of cytokines into the blood stream. This side effect is called cytokine release syndrome or tumor lysis syndrome (TLS) and can be severe or even life threatening for the patient. A TLS is observed in any CAR treatment against a cancer, regardless of the LCAR/CAR pairing itself. The invention focuses on the disturbance of the formation of a LCAR/CAR binding axis. It is achieved by the introduction of LCAR into a VLP, which in turn binds preferentially to a CAR without activating it. The invention represents, for the first time, a reversible in vivo control system for CAR T cells that leaves the CAR T cells intact and thus, may prevent TLS and related effects. Thus, the invention allows to fine tune the CAR T cell response.

### SUMMARY OF THE INVENTION

In a first aspect the invention relates to a VSP, wherein: (i) the VSP is capable of, optionally together with one or more further VSPs and optionally phospholipids, forming a capsomeric structure, a capsid, a virus like particle (VLP), a viral vector or a virus, (ii) the VSP comprises in a region that is located on the surface of said capsomeric structure, capsid, VLP, viral vector or virus at least one ligand specifically binding to a chimeric antigen receptor (LCAR), and wherein the LCAR is a polypeptide.

In a second aspect the invention further relates to a nucleic acid encoding the VSP of the first aspect.

In a third aspect the invention further relates to a capsomeric structure, a capsid, a VLP, a viral vector or a virus comprising at least one VSP according to the first aspect of the invention.

In a fourth aspect the invention relates to a pharmaceutical composition comprising the VSP of the first aspect, the nucleic acid of the second aspect, the capsomeric structure, a capsid, a VLP, a viral vector or a virus comprising at least one VSP according to the third aspect of the invention.

A fifth aspect of the invention relates to a VSP, a nucleic acid, a capsomeric structure, a capsid, a VLP, a viral vector or a virus according to the first, second and third aspect for use in medicine.

A sixth aspect of the invention relates to a VSP, a nucleic acid, a capsomeric structure, a capsid, a VLP, a viral vector or a virus according to the first, second or third aspect for use in decreasing or limiting an immune response, preventing, decreasing or limiting an immune response, preferably elicited by an adoptive immune therapy, more preferably by CAR cell therapy.

In a seventh aspect the present invention relates to a kit of parts comprising a capsomeric structure, a capsid, a VLP, a viral vector or a virus comprising at least one VSP according to the first aspect of the invention, wherein the viral vector or virus is non-infectious and a modified cell expressing a CAR, that is specifically bound by said capsomeric structure, said capsid, said VLP, said viral vector or said virus.

### LIST OF FIGURES

In the following, the content of the figures comprised in this specification is described. In this context please also refer to the detailed description of the invention above and/or below.

FIG. 1: ELISA of recombinant AAV particles. 1×10⁸ viral particles of wt and NY-BR1 AAV per well were coated and detection was performed by incubation with AAV-specific A20 mouse hybridoma supernatant in 2-fold serial dilution, followed by anti-mouse IgG-HRP antibody. DAB served as substrate and OD450 was measured. Experiment was done in triplicate.

FIG. 2: ELISA of recombinant AAV particles. 1×10⁸ viral particles of wt and NY-BR1 AAV per well were coated in 2-fold serial dilution, detection was performed by incubation with NY-BR1 specific mouse antibody Morab2 (1 g/ml), followed by anti-mouse IgG-HRP antibody. DAB served as substrate and OD450 was measured. Experiment was done in triplicate.

FIG. 3: ELISA of recombinant AAV particles. 1×10⁸ viral particles of NY-BR1 AAV per well were coated and detection was performed by incubation with either AAV-specific A20 mouse hybridoma supernatant or NY-BR1 specific scFv-Fc fusion protein each in 2-fold serial dilution, followed by the respective anti-mouse or anti-human IgG-HRP antibody. DAB served as substrate and OD450 was measured. Experiment was done in triplicate.

FIG. 4: Example of CAR down regulation in a cell line. Jurkat cells stably expressing a NY-BR1 specific CAR were incubated with wt or NY-BR1 AAV carrying a GFP expression cassette at MOI 5000 for 24 h. CAR expression was detected by FACS using an anti-human IgG APC antibody in addition to GFP transgene expression.

FIG. 5: Time course analysis of the experiment shown in FIG. 4 with respect to transgene and CAR expression. Respective AAV particles were present in culture medium over time. Experiment was done in triplicate, error bars show SEM.

FIG. 6: FACS analysis of CAR expression over time as in FIGS. 4 and 5 but AAV particles were removed from culture medium 4 h post transduction. Experiment was done in triplicate, error bars show SEM.

FIG. 7: Activation assay of Jurkat cells expressing NY-BR1 CAR. Expression level of the activation marker CD69 was measured by FACS after transduction with respective AAV particles at MOI 5000 for 24 h compared to non-transduced cells and PMA-Ionomycin-stimulated cells.

FIG. 8: Example of CAR down regulation in primary human CD3+ cells (donor 3) assessed by FACS (experimental conditions as in FIG. 4) and measurements of T cell activation of three different donors by the use of IFNgamma ELISA kit.

FIG. 9: Example of CAR down regulation independent of AAV serotype. Insert NY-BRIdisplayed at position 588 in AAV2 and analogous positions in AAV9 and AAV5 and in a position proximal to the analogous position in AAV8. Jurkat cells stably expressing a NY-BR1-specific CAR were incubated with wt AAV2 or NY-BR1 AAVs carrying a GFP expression cassette at MOI 5000 for 24 h. CAR expression was detected by FACS using an anti-human IgG APC antibody. Experiment was done in triplicate, error bars show SEM.

FIG. 10: Assessment ofNY-BR1 LCAR length variation. Streptavidin-coated Dynabeads were coupled with A20-biotin followed by incubation with crude lysates of AAVs displaying various lengths of NY-BR1 LCAR. Quantification of binding to soluble CAR was performed by FACS using NY-BR1 specific scFv-Fc fusion protein and secondary antibody anti-hu-IgG-PE; ELISA of intact AAV particles. AAV-specific A20 mouse hybridoma supernatant was coated followed by incubation with AAV crude lysates. Then, biotin-conjugated A20 was added followed by STAV-HRP conjugate. DAB served as substrate and OD450 was measured.

| AAV# | Sequence |
|---|---|
| 150 | Insert NYBR1 |
| 285 | Insert NYBR1_1-4 |
| 286 | Insert NYBR1_3-6 |
| 287 | Insert NYBR1_5-8 |
| 288 | Insert NYBR1_7-10 |
| 289 | Insert NYBR1_9-12 |
| 290 | Insert NYBR1_11-14 |
| 309 | Insert NYBR1_1-8 |
| 310 | Insert NYBR1_4-11 |
| 311 | Insert NYBR1_7-14 |
| 345 | Insert NYBR1_20 |

FIG. 11: Specific inhibition of CAR-mediated killing from 10-20 h post addition of CAR T cells. Primary breast cancer cells expressing NY-BR1 were co-cultivated with NY-BR1 specific CAR T cells in an effector to target ratio of 1:1 and with or without NY-BR-LCAR AAV particles (MOI 5000). Target cell viability was recorded on a real time impedance measurement device (xCelligence, ACEA Biosystems Inc.) and is shown as mean viability with SEM.

FIG. 12: Shows an alignment of VP sequences of common AAV serotypes. Multiple sequence alignment was performed by Clustal Omega and displayed by MView 1.63. Preferred insertion points M1, P34, T138, A139, K161, S261, A266, N381, R447, T448, G453, R459, R471, F534, T573, Q584, N587, R588, A591, P657, A664, T713 or T716 in AAV2 are highlighted by bold print and underline. All aligned serotypes show stretches of high almost 100% sequence homology with each other and stretches of lower homology. There are also gaps in the alignment with the consequence that the absolute amino acid positions of amino acid stretches with high sequence similarity or of a particular amino acid within such a stretch are different among different serotypes. Thus, amino acids of AAV at analogous positions to, e.g. arginine at the absolute position 588 of AAV2, i.e. R588 (when counting from the first N-terminal amino acid of AAV2 VP1) are at absolute amino acids position T578 in AAV5, at position T591 in AAV8, and at position A589 in AAV9, The actual insertion points for the LCAR NY-BR1 tested in Example 6 (see FIG. 9) are highlighted by a grey underlay. The alignment indicates a numbering of amino acids 1 to 761 which are referred to in the context of the present invention as Adeno Associated Virus Homology Position (AAHP) based on all seventeen aligned AAV sequences. For example R588 of AAV2 has an AAHP of 614. The amino acid of VP1 after which NY-BR1 LCAR was inserted C-terminally into AAV2, AAV5, AAV8 and AAV9, respectively, are indicated by grey rectangles. Accordingly, with reference to AAHP NY-BR1 LCAR was inserted C-terminally of AAHP 614 in AAV2, AAV5 and AAV9 and C-terminally of AAHP 613 in AAV8.

FIG. 13: Example of wt and NY-BR1 AAV2 binding to Jurkat cells with and without NY-BR1 CAR in the presence or absence of heparin. AAVs at 1E5 capsids/cell were pre-incubated with heparin, then, Jurkat cells were added and the mixture was incubated on ice for 1 h. Bound AAV was quantified by staining with biotin-conjugated anti-capsid antibody A20 and STAV-Alexa488. Alexa488-positive living cells were detected by FACS analysis. Experiment was done in triplicate, error bars show SEM.

### LIST OF SEQUENCES

SEQ ID NO: 1 VP1 Viral coat protein 1 of AAV serotype 2
SEQ ID NO: 2 VP2 Viral coat protein 2 of AAV serotype 2
SEQ ID NO: 3 VP3 Viral coat protein 3 of AAV serotype 2

### SEQ ID NO: 4 Sequence of VP1 of AAV2-NY-BR1

### SEQ ID NO: 5 Sequence of VP1 of AAV5-NY-BR1

### SEQ ID NO: 6 Sequence of VP1 of AAV8-NY-BR1

### SEQ ID NO: 7 Sequence of VP1 of AAV9-NY-BR1

SEQ ID NO: 8 Sequence of insert NYBR1_20
SEQ ID NO: 9 Sequence of insert NYBR1
SEQ ID NO: 10 Sequence of insert NYBR1_1-4
SEQ ID NO: 11 Sequence of insert NYBR1_3-6
SEQ ID NO: 12 Sequence of insert NYBR1_5-8
SEQ ID NO: 13 Sequence of insert NYBR1_7-10
SEQ ID NO: 14 Sequence of insert NYBR1_9-12
SEQ ID NO: 15 Sequence of insert NYBR1_11-14
SEQ ID NO: 16 Sequence of insert NYBR1_1-8
SEQ ID NO: 17 Sequence of insert NYBR1_4-11
SEQ ID NO: 18 Sequence of insert NYBR1_7-14
SEQ ID NO: 19 VP1 Viral coat protein 1 of AAV serotype 1
SEQ ID NO: 20 VP1 Viral coat protein 1 of AAV serotype 3a
SEQ ID NO: 21 VP1 Viral coat protein 1 of AAV serotype 3b
SEQ ID NO: 22 VP1 Viral coat protein 1 of AAV serotype 4
SEQ ID NO: 23 VP1 Viral coat protein 1 of AAV serotype 5
SEQ ID NO: 24 VP1 Viral coat protein 1 of AAV serotype 6
SEQ ID NO: 25 VP1 Viral coat protein 1 of AAV serotype 6.2
SEQ ID NO: 26 VP1 Viral coat protein 1 of AAV serotype 7
SEQ ID NO: 27 VP1 Viral coat protein 1 of AAV serotype 8
SEQ ID NO: 28 VP1 Viral coat protein 1 of AAV serotype 9
SEQ ID NO: 29 VP1 Viral coat protein 1 of AAV serotype 10
SEQ ID NO: 30 VP1 Viral coat protein 1 of AAV serotype 11
SEQ ID NO: 31 VP1 Viral coat protein 1 of AAV serotype 12
SEQ ID NO: 32 VP1 Viral coat protein 1 of AAV serotype 13
SEQ ID NO: 33 VP1 Viral coat protein 1 of AAV serotype.rh10
SEQ ID NO: 34 VP1 Viral coat protein 1 of AAV serotype.rh32.33 DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “viral coat protein” (VCP) as used in the context of the present invention refers to a structural virus capsid protein of a virus. Preferably the virus is a double-stranded DNA virus, single-stranded DNA virus, double-stranded RNA virus, single-stranded RNA virus, negative-sense single-stranded RNA virus, single-stranded RNA reverse transcribing virus, double-stranded RNA reverse transcribing virus. The VCP can comprise major capsid proteins of adeno-associated virus (AAV) such as VP1, VP2 or VP3 alone or in combination. VP1, VP2 and VP3 may interact together to form a higher order structure with icosahedral symmetry.

The term “viral structural proteins” (VSP) is used in the context of the present invention to refer to viral coat proteins or viral envelope glycoproteins.

The term “viral envelope glycoproteins” (VEG) is used in the context of the present invention to refer to viral proteins that are part of the viral envelope. The viral envelope is typically derived from portions of the host cell membrane, e.g. comprises phospholipids, and additionally comprise viral glycoproteins that, e.g. help the virus to avoid the immune system. Enveloped viruses comprise DNA viruses, in particular Herpesviruses, Poxviruses, and Hepadnaviruses; RNA viruses, in particular Flavivirus, Togavirus, Coronavirus, Hepatitis D, Orthomyxovirus, Paramyxovirus, Rhabdovirus, Bunyavirus, Filovirus and Retroviruses. Accordingly, the viral envelop glycoprotein is preferably derived from any of these viruses.

The term “capsid” or “capsids” as used in the context of the present invention refers to a three-dimensional structure of single or double protein shells of non-covalently linked multimers of only one type or two, three or more different types of VCPs. The VCPs self-assemble to form the capsid. Usually self-assembly of virus capsids follows two basic patterns: helical symmetry, in which the protein subunits and the nucleic acid are arranged in a helix, and icosahedral symmetry, in which the protein subunits assemble into a symmetric shell that covers the nucleic acid-containing core.

The term “capsomeric structure” as used herein refers to a structure formed of only one type or two, three or more different types of VCPs depending on the particular virus of which the VCP is derived from. The coat of adenovirus, for example, comprises the three coat proteins hexon, penton and fibre. However, penton alone is capable of forming a capsomeric structure if independently expressed. The coat of AAV, for example, comprises three coat proteins VP1, VP2 and VP3. However, VP3 alone is capable of forming a capsomeric structure if independently expressed. Nevertheless, capsomeric structures can bind to cell surfaces and can be internalized by cells to which they bind. Capsomeric structures may occur in nature or may be artificial. Typically, capsomeric structures do not comprise viral nucleic acids and are, thus, non-infectious.

The term “vector” is used in the context of the present invention to refer to a polynucleotide or one or more proteins or a mixture thereof that can be used to transport the polynucleotide comprising one or more genes encoding, e.g. a gene product of interest or a RNA, in particular a miRNA, or siRNA, or a protein of interest into a suitable host or target cell. Examples of vectors include but are not limited to plasmids, cosmids, phages, viruses or artificial chromosomes. Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences comprise but are not limited to promoters, enhancers, silencers, insulators, or repressors. In a vector comprising more than one polynucleotide encoding one or more gene products of interest, the expression may be controlled together or separately by one or more expression control sequences. More specifically, each polynucleotide comprised in the vector may be controlled by a separate expression control sequence or all polynucleotides comprised in the vector may be controlled by a single expression control sequence. Polynucleotides comprised in a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The term “viral vector” as used in the context of the present invention refers to a virus that is modified to transfer a polynucleotide or a given protein comprised in the viral vector into a target cell. The use of viral vectors is preferred in the context of the present invention.

A virus like particle (VLP) is a multimer of VSP, preferably of VCPs and/or VEPs that does not comprise polynucleotides but which otherwise has properties of a virus, e.g. binds to cell surface receptors, is internalized with the receptor, is stable in blood, and/or comprises glycoproteins etc. VLPs are typically assembled of multimers of VCPs and/or VEPs, in particular of VCPs. VLPs are well known in the art and have been produced from a number of viruses including Parvoviridae (e.g. adeno-associated virus), Retroviridae (e.g. HIV), Flaviviridae (e.g. Hepatitis C virus) and bacteriophages (e.g. Qβ, AP205).

The term “VP1”, “VP2” and “VP3” as used in the context of the present invention refers to the VCP VP1, VP2 and VP3. VP1, VP2 and VP3 are viral capsid proteins, preferably of AAV, which self-assemble to form an icosahedral capsid with a T=1 symmetry, about 22 nm in diameter. Preferably, the thus assembled capsid consists of 60 copies of three size variants VP1, VP2 and VP3 in a 1:1:10 ratio, e.g. from adeno-associated virus serotype 2. The three size variants of the capsid protein VP1, VP2 and VP3 differ in their N-terminus, i.e. VP2 and VP3 are truncated forms of VP1. The capsid encapsulates the genomic DNA or RNA, single or double-stranded depending on the virus. In naturally occurring AAV, the capsid encapsulates a single-stranded DNA.

AAV capsid proteins bind to host cell heparan sulfate proteoglycan and use host co-receptors such as αVβ5 integrin to provide virion attachment to the target cell. This attachment induces virion internalization predominantly through clathrin-dependent endocytosis. Binding to the host receptor also induces capsid rearrangements leading to surface exposure of the VP1 N-terminus, specifically its phospholipase A2-like region and putative nuclear localization signal(s). The VP1 N-terminus might serve as a lipolytic enzyme to breach the endosomal membrane during entry into host cell and might contribute to virus transport to the nucleus.

The term “virus” as used herein refers to small obligate intracellular parasites, which by definition contain either a RNA or DNA genome surrounded by a protective protein coat, i.e. a capsid. The genome of a virus may consist of DNA or RNA, which may be single stranded (ss) or double stranded (ds), linear or circular. The entire genome may occupy either one nucleic acid molecule (monopartite genome) or several nucleic acid segments (multipartite genome). The virus may comprise double-stranded DNA virus, preferably Myoviridae, Siphoviridae, Podoviridae, Herpesviridae, Adenoviridae, Baculoviridae, Papillomaviridae, Polydnaviridae, Polyomaviridae, Poxviridae; single-stranded DNA virus, preferably Anelloviridae, Inoviridae, Parvoviridae; double-stranded RNA virus, preferably Reoviridae; single-stranded RNA virus, preferably Coronaviridae, Picornaviridae, Caliciviridae, Togaviridae, Flaviviridae, Astroviridae, Arteriviridae, Hepeviridae; negative-sense single-standed RNA virus, preferably Arenaviridae, Filoviridae, Paramyxoviridae, Rhabdoviridae, Bunyaviridae, Orthomyxoviridae, Bornaviridae; single-stranded RNA reverse transcribing virus, preferably Retroviridae; double-stranded RNA reverse transcribing virus, preferably Caulimoviridae, Hepadnaviridae.

The term “adeno-associated virus” (AAV) refers to a virus belonging to the family of Parvoviridae, containing several genera which can be subdivided into the family of Parvovirinae comprising Parvovirus, Erythrovirus, Dependovirus, Amdovirus and Bocavirus and the family of Densoviriniae comprising Densovirus, Iteravirus, Brevidensovirus, Pefudensovirus and Contravirus. The unique life cycle of AAV and its ability to infect both non-dividing and dividing cells with persistent expression have made it an attractive vector. An additional attractive feature of the wild-type virus is the lack of apparent pathogenicity.

The term “specific binding” as used in the context of the present invention means that the ligand binds with a higher affinity to its respective target than to any other target. Ligands bind with a certain affinity to their targets and the binding of the ligand to its respective target, for example a receptor protein typically results in a biological effect. Preferably, the binding of the ligand to its target is both specific and occurs with a high affinity, preferably with K_{d} of less than 10⁻⁷, 10⁻⁸, 10⁻⁹, 10⁻¹⁰ M or less. Such affinity is preferably measured at 37° C. Suitable assays include surface plasmon resonance measurements (e.g. Biacore), quartz crystal microbalance measurements (e.g. Attana), and competition assays.

The term“chimeric antigen receptor” (CAR; also known as chimeric immunoreceptor, chimeric T cell receptor, artificial T cell receptor) refers to engineered receptors, which graft an arbitrary specificity onto an immune effector cell, preferably a T cell. Cells are genetically equipped with a CAR, which is a composite membrane receptor molecule and provides both targeting specificity and T cell activation. The most common form of CARs are fusions of single chain variable fragment (scFv) derived from monoclonal antibodies, fused to CD3 transmembrane- and endodomain. The CAR targets the T cell to a desired cellular target through an antibody-derived binding domain in the extracellular moiety, and T cell activation occurs via the intracellular moiety signalling domains when the target is encountered. The transfer of the coding sequence of these receptors into suitable cells, in particular T cells, is commonly facilitated by retro- or lentiviral vectors. The receptors are called chimeric because they are composed of parts from different sources.

The term “ligand to chimeric antigen receptor” (LCAR) in the context of the present invention relates to a polypeptide, preferably comprising, essentially comprising or consisting of at least one, two, three or more surface exposed epitopes of a TSEA that is (are) specifically bound by a CAR or a polypeptide that can specifically bind to the CAR, e.g. a CAR specific antibody, in particular a scFv, an antibody-like protein or fragment thereof. The LCAR is typically that part of a protein or glycoprotein that forms a conformational or non-conformational epitope accessible on the surface of the target cell targeted by CAR cell therapy. Since CAR therapy is primarily targeted against tumor diseases the LCAR preferably comprises one or more epitope(s) of a tumor-specific or tumor-associated antigen. The polypeptide can be inserted into a VSP, in particular a VCP or VEP. Further, binding of the LCAR to its CAR results in a temporary unavailability of the CAR on the cell surface. Since CARs often comprise scFv, the length of the LCAR is preferably at least the length of a B cell epitope. In a preferred embodiment the LCAR does not comprise one or more T cell epitopes, although T cell epitopes only elicit a T cell response in complex with MHC class I or II. The skilled person is well aware how to assess whether a given protein is capable of eliciting a T cell response in the context of MHC I and MHC II presentation. These methods include in silico T-cell epitope prediction (see, e.g. Desai DV and Kulkarni-Kale U (2014) Methods Mol. Biol. 1184:3333-364 and Kosaloglu et al., (2016) Oncoimmunology) and techniques such as the ELISPOT assay, intracellular cytokine staining or Tetramer/Pentramer stainings followed by flow cytometry. It is preferred that the LCAR does not elicit a T-cell immune response because in this way unwanted T cell responses against the virus of the invention are prevented. It is further preferred that LCAR does not elicit a B cell response. While the CAR comprises a single chain antibody that specifically binds to one or more TSEAs, the LCAR should not be capable of inducing a B cell response, i.e. be immunogenic, in as long as it is capable to specifically bind to the CAR. Thus, a LCAR that neither elicits a T nor a B cell response is particularly preferred since it avoids unwanted immune responses against the virus of the invention when administered to a patient.

The term “tumor-surface exposed antigen” (TSEA) refers to the product of an oncofetal gene, which is typically only expressed in fetal tissues and in cancerous somatic cells; a product of an oncoviral gene, which is encoded by tumorigenic transforming viruses; a product of an overexpressed/accumulated gene, which is expressed by both normal and neoplastic tissue, with the level of expression highly elevated in neoplasia; a product of a cancer-testis gene, which is expressed only by cancer cells and adult reproductive tissues such as testis and placenta; a product of a lineage-restricted gene, which is expressed largely by a single cancer histotype; a product of a mutated gene, which is only expressed by cancer as a result of genetic mutation or alteration in transcription, and a post-translationally altered product, which comprises, e.g. tumor-associated alterations in glycosylation; or a product of an idiotypic gene, which is highly polymorphic and where a tumor cell expresses a specific “clonotype”, e.g. as in B cell, or T cell lymphoma/leukemia resulting from clonal aberrancies; that comprises epitopes present on the surface of tumor cells. Preferably, the TSEA is preferentially or exclusively expressed in tumor cells. Preferred are those tumor-surface exposed antigens that are the target of CAR therapy. Preferably, such antigens are proteins or glycoproteins. Above definition of TSEA covers both tumor specific antigens and tumor associated antigens. An epitope is present or “exposed” on the surface of a tumor cell, if a cell of the immune system, e.g. a T cell, a B cell, an immune cell modified with a CAR, in particular a CAR T cell or an antibody, e.g. IgA, IgG, or IgE, can specifically bind to the TSEA by binding to an epitope of the TSEA on the surface of the tumor cell.

An “epitope”, also known as antigenic determinant, is used in the context of the present invention to refer to the segment of a macromolecule that is recognized by the immune system, specifically by antibodies, B cells, or T cells. Such epitope is that part or segment of a macromolecule capable of binding to an antibody or antigen-binding fragment thereof. In this context, the term “binding” preferably relates to a specific binding. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. An epitope may be immunogenic in itself, e.g. trigger a B-cell response or may just be capable of specifically binding to an antibody.

As used herein, a “conformational epitope” refers to an epitope of a linear macromolecule (e.g. a polypeptide) that is formed by the three-dimensional structure of said macromolecule. In the context of the present application, a “conformational epitope” is a “discontinuous epitope”, i.e. the conformational epitope on the macromolecule (e.g. a polypeptide) which is formed from at least two separate regions in the primary sequence of the macromolecule (e.g. the amino acid sequence of a polypeptide). In other words, an epitope is considered to be a “conformational epitope” in the context of the present invention, if the epitope consists of at least two separate regions in the primary sequence to which the specifically binding part of a CAR, e.g. an antibody, single chain antibody or an antigen-binding fragment thereof binds simultaneously, wherein these at least two separate regions are interrupted by one more region in the primary sequence to which the specifically binding part of a CAR does not bind. In particular, such a “conformational epitope” is present on a polypeptide, and the two separate regions in the primary sequence are two separate amino acid sequences to which the specifically binding part of a CAR binds.

The term “specifically binding” refers to the fact that the member of a binding pair interacts with a higher affinity with its binding partner than with other binding partners. The “binding affinity” between two binding partners is determined by the strength of the sum of non-covalent interactions between a single binding site of a molecule (e.g., CAR) and its binding partner (e.g., LCAR). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). “Specific binding” means that a binding moiety (e.g. an antibody) binds stronger to a target such as an epitope for which it is specific compared to the binding to another target. A binding moiety binds stronger to a first target compared to a second target if it binds to the first target with a dissociation constant (Kd) which is lower than the dissociation constant for the second target. The dissociation constant (Kd) for the target to which the binding moiety binds specifically is more than 10-fold, preferably more than 20-fold, more preferably more than 50-fold, even more preferably more than 100-fold, 200-fold, 500-fold or 1000-fold lower than the dissociation constant (Kd) for the target to which the binding moiety does not bind specifically.

Accordingly, the term “Kd” (measured in “mol/L”, sometimes abbreviated as “M”) is intended to refer to the dissociation equilibrium constant of the particular interaction between a binding moiety (e.g. an antibody or fragment thereof) and a target molecule (e.g. an antigen or epitope thereof). Affinity can be measured by common methods known in the art, including but not limited to surface plasmon resonance based assay (such as the BIAcore assay); quartz crystal microbalance assays (such as Attana assay); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

Preferably, the VSPs of the present invention, in particular if assembled into a capsomeric structure, a capsid, a VLP, a viral vector or a virus of the present invention bind to a CAR, in particular if expressed on T-cells, with a binding affinity that allows endocytosis and, thus reduction of the cellular concentration of the CAR on a T-cell. This property can be tested for each VSPs of the present invention, in particular if assembled into a capsomeric structure, a capsid, a VLP, a viral vector or a virus of the present invention and a given CAR, i.e. the CAR that is specifically bound, by following the teaching in the examples. Preferably, the affinity (Kd) between a VSP of the present invention, in particular if assembled into a capsomeric structure, a capsid, a VLP, a viral vector or a virus of the present invention and the CAR that is specifically bound is between 10 μM to 1 pM, preferably less than 10 μM, 5 μM, 1 μM, 500 nM, 450 nM, 400 nM, 350 nM, 300 nM, 250 nM, 200 nM, 150 nM, 100 nM, 50 nM, 10 nM, 1 nM, 900 pM, 800 pM, 700 pM, 600 pM, 500 pM, 400 pM, 300 pM, 200 pM, 100 pM, 50 pM, or 1 pM.

The terms “protein” and “polypeptide” are used interchangeably herein and refer to any peptide-bond-linked chain of amino acids, regardless of length or post-translational modification. Proteins usable in the present invention (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5th ed., Freeman and Company (2002). Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12(13):R464-R466). The term “amino acid” also includes unnatural amino acids, modified amino acids (e.g., having modified side chains and/or backbones), and amino acid analogs. See, e.g., Zhang et al. (2004) “Selective incorporation of 5-hydroxytryptophan into proteins in mammalian cells,” Proc. Natl. Acad. Sci. U.S.A. 101(24):8882-8887, Anderson et al. (2004) “An expanded genetic code with a functional quadruplet codon” Proc. Natl. Acad. Sci. U.S.A. 101(20):7566-7571, Ikeda et al. (2003) “Synthesis of a novel histidine analogue and its efficient incorporation into a protein in vivo,” Protein Eng. Des. Sel. 16(9):699-706, Chin et al. (2003) “An Expanded Eukaryotic Genetic Code,” Science 301(5635):964-967, James et al. (2001) “Kinetic characterization of ribonuclease S mutants containing photoisomerizable phenylazophenylalanine residues,” Protein Eng. Des. Sel. 14(12):983-991, Kohrer et al. (2001) “Import of amber and ochre suppressor tRNAs into mammalian cells: A general approach to site-specific insertion of amino acid analogues into proteins,” Proc. Natl. Acad. Sci. U.S.A. 98(25):14310-14315, Bacher et al. (2001) “Selection and Characterization of Escherichia coli Variants Capable of Growth on an Otherwise Toxic Tryptophan Analogue,” J. Bacteriol. 183(18):5414-5425, Hamano-Takaku et al. (2000) “A Mutant Escherichia coli Tyrosyl-tRNA Synthetase Utilizes the Unnatural Amino Acid Azatyrosine More Efficiently than Tyrosine,” J. Biol. Chem. 275(51):40324-40328, and Budisa et al. (2001) “Proteins with {beta}-(thienopyrrolyl) alanines as alternative chromophores and pharmaceutically active amino acids,” Protein Sci. 10(7):1281-1292. Amino acids can be merged into peptides, polypeptides, or proteins.

The term“insertion site” as referred to in the context of the present application means the exact amino acid position in the respective VCP wherein the LCAR polypeptide sequence is inserted, e.g. the inserted amino acid sequence can simply be inserted between two given amino acids of the structural VCP leading to the addition of amino acids to the original amino acid sequence of the VCP. Different scenarios may concomitantly occur by inserting a polypeptide sequence between two given amino acids, such as deletions of amino acids present in the original VCP amino acid sequence, leading to a complete substitution or partial substitution of the given amino acid of the VCP.

The term “variant” is to be understood as a polypeptide or polynucleotide which differs in comparison to the polypeptide or polynucleotide from which it is derived by one or more changes in its length or sequence. The polypeptide or polynucleotide from which a polypeptide or polynucleotide variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed are modified molecules such as but not limited to post-translationally modified proteins (e.g. glycosylated, biotinylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Typically, a variant is constructed artificially, preferably by gene-technological means, whilst the parent protein or polynucleotide is a wild-type protein or polynucleotide, or a consensus sequence thereof. However, also naturally occurring variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, i.e. is functionally active.

In particular, the term “peptide variant”, “polypeptide variant”, “protein variant” is to be understood as a peptide, polypeptide, or protein which differs in comparison to the peptide, polypeptide, or protein from which it is derived by one or more changes in the amino acid sequence. The peptide, polypeptide, or protein, from which a peptide, polypeptide, or protein variant is derived, is also known as the parent peptide, polypeptide, or protein. Further, the variants usable in the present invention may also be derived from homologs, orthologs, or paralogs of the parent peptide, polypeptide, or protein or from artificially constructed variant, provided that the variant exhibits at least one biological activity of the parent peptide, polypeptide, or protein. The changes in the amino acid sequence may be amino acid exchanges, insertions, deletions, N-terminal truncations, or C-terminal truncations, or any combination of these changes, which may occur at one or several sites. A peptide, polypeptide, or protein variant may exhibit a total number of up to 60 (up to 1, 2, 3, 4, 5, 6, 7, 8, 9, 10, 15, 20, 25, 30, 35, 40, 45, 50, 55, 60) changes in the amino acid sequence (i.e. exchanges, insertions, deletions, N-terminal truncations, and/or C-terminal truncations). The amino acid exchanges may be conservative and/or non-conservative. Alternatively or additionally, a “variant” as used herein, can be characterized by a certain degree of sequence identity to the parent peptide, polypeptide, or protein from which it is derived. More precisely, a variant in the context of the present invention exhibits at least 80% sequence identity, more preferably at least 85% sequence identity, even more preferably at least 90% sequence identity, and most preferably at least 95% sequence identity to the reference polypeptide. Preferably, the variants of the present invention exhibit the indicated sequence identity, and preferably the sequence identity is over a continuous stretch of 15, 20, 25, 30, 35, 40, 45 or 50 or more amino acids. Most preferably the indicated identity is determined over the entire length of the alignment between the two amino acids, i.e. the reference amino acid and the amino acid that is assessed for its identity. Such amino acid sequence alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/or http://www.ebi.ac.uk/Tools/clustalw2/index.html. The grade of sequence identity (sequence matching) may be calculated using e.g. BLAST, BLAT or BlastZ (or BlastX). Preferably, sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

The “percentage of sequences identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” in the context of two or more nucleic acids or polypeptide sequences, refers to two or more sequences or subsequences that are the same, i.e. comprise the same sequence of nucleotides or amino acids. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over a specified region), when compared and aligned for maximum correspondence over a comparison window, or designated region as measured using one of the above sequence comparison algorithms or by manual alignment and visual inspection. These definitions also refer to the complement of a test sequence. Accordingly, the term “at least 80% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

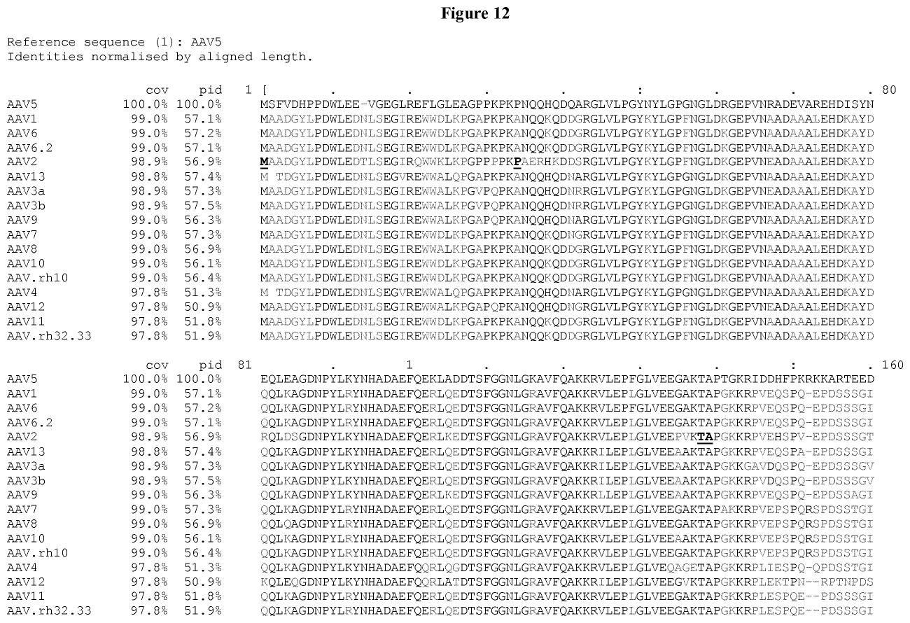

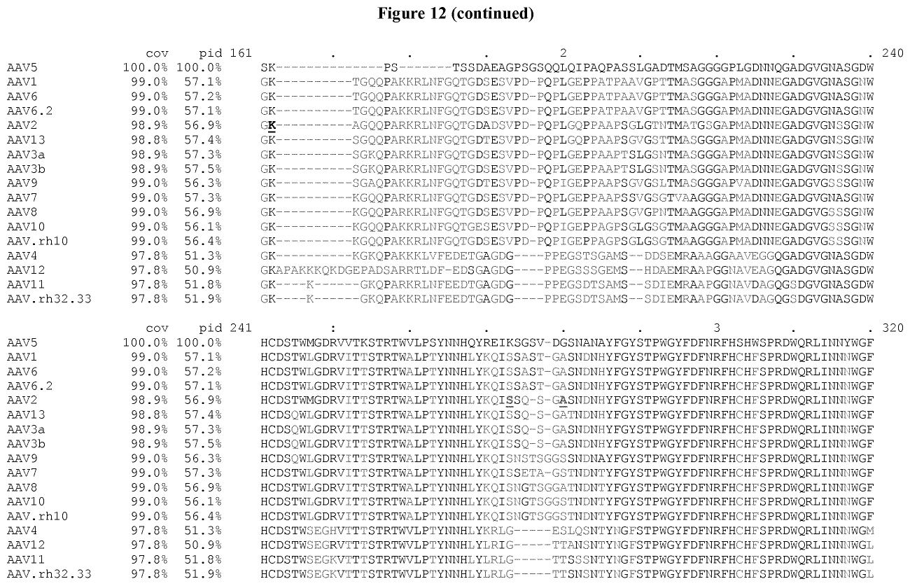

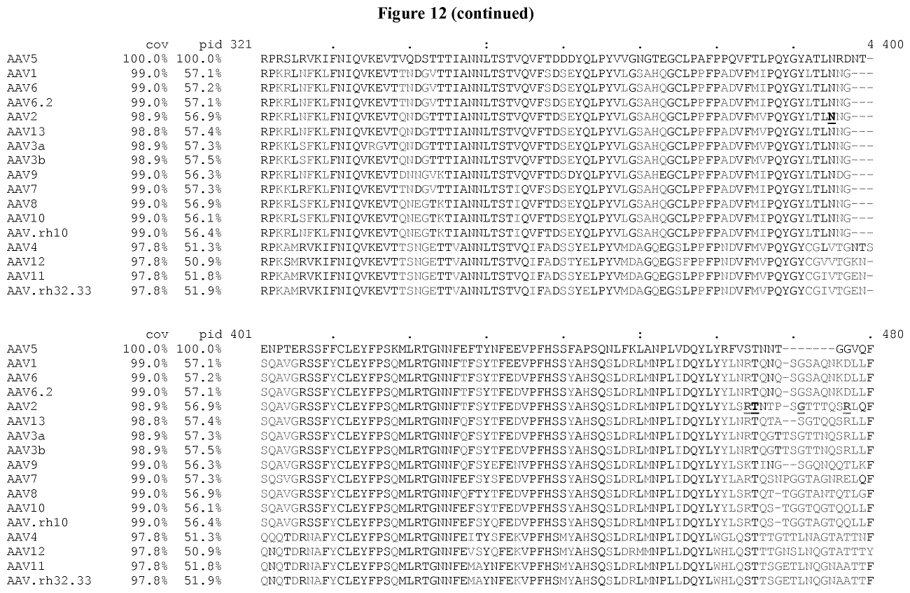

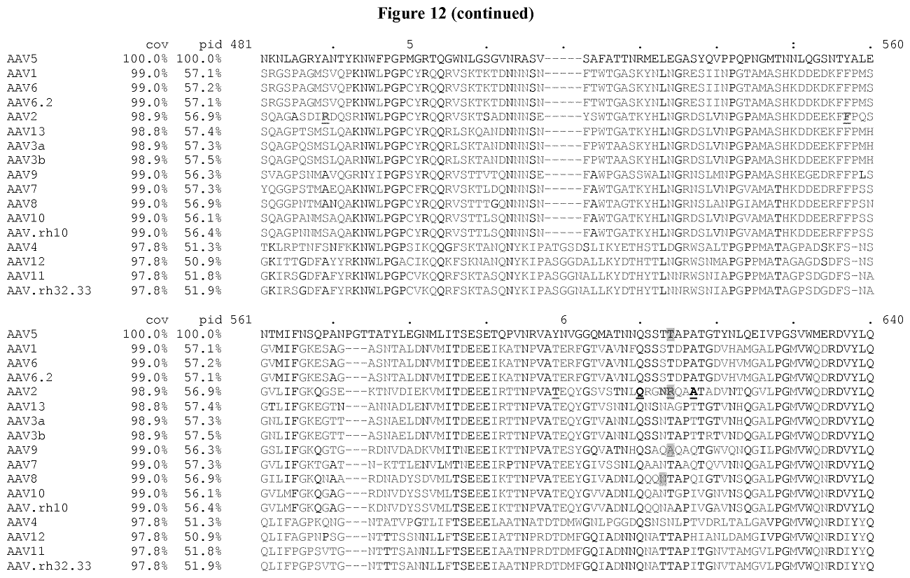

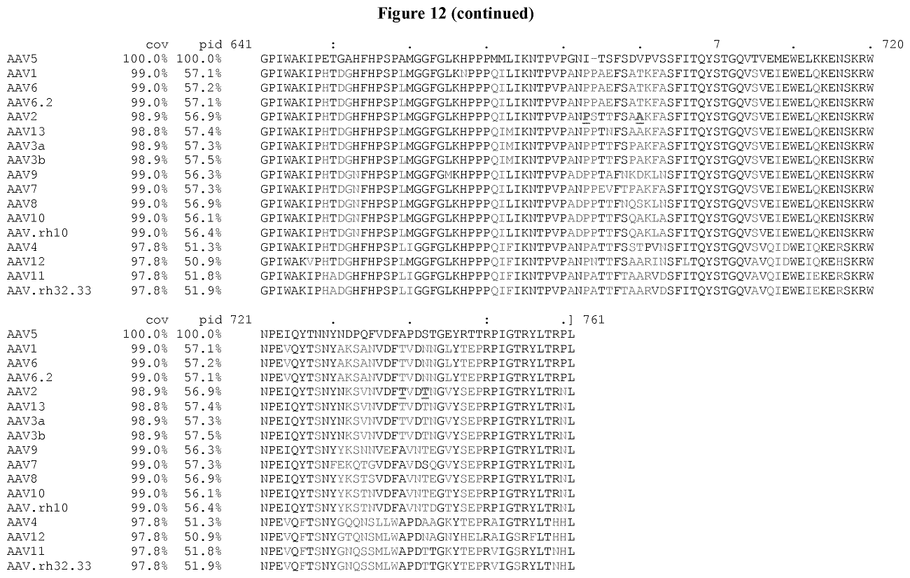

The term “analogous position” of a VSP as used in the context of the present invention to refer to an amino acid that is at the same relative position within a VSP as that of another reference VSP, if the two proteins are aligned by using an alignment algorithm, i.e. that align with each other or that is proximal to an amino acid with the same relative position. A commonly used alignment algorithm is Clustal Omega which is publically accessible at the EMBL-EBI website (https://www.ebi.ac.uk/Tools/msa/clustalo/). Related VSPs, in particular of viruses of the same species, e.g. Adeno-associated virus, typically show stretches of high and low sequence homology. The extent of the sequence variation is typically dependent on whether the respective sequence is essential for the protein or not. Thus, if VP1 of AAV2 is used as the reference VSP and one or more other AAV VSPs are aligned with VP1 of AAV2, the skilled person can readily determine analogous positions in VP1 of other AAVs to a given amino acid of VP1 of AAV2. This is exemplarily demonstrated in FIG. 12 which depicts an alignment of 17 different AAV serotypes and highlights several amino acids of AAV2 by bold print and underline that are taught in the context of the present invention to be particularly suitable for the insertion of LCARs. The amino acids aligning with the highlighted amino acids of AAV2 are at an analogous position. With respect to AAV VP1 protein, the Adeno-Associated Virus Homology Position (AAHP) is also used to refer to an insertion point within an AAV VP1. The AAHP is the number given to the aligned amino acids based on the length of the overall alignment, which will comprise gaps. For example R588 of AAV2 has an AAHP of 614 in FIG. 12. Proximal positions that are preferably included in this definition are amino acids at position 1 or 2 amino acids N-terminal or 1 or 2 amino acids C-terminal to the amino acid position that has the same relative position, e.g. N590 of AAV8 is proximal to T591 of AAV8 and, thus in a preferred embodiment also considered at an analogous position to R588 of AAV2 (which aligns with T591 of AAV8, T578 of AAV5 and A589 of AAV9).

The term “fragment” used herein refers to naturally occurring fragments (e.g. splice variants) as well as artificially constructed fragments, in particular to those obtained by gene-technological means. Typically, “fragments” are smaller in length or size than the parental molecule. “Fragments” refer to a smaller part of peptides, polypeptides or proteins in size and length than the parental molecule but which is still as functional as the parental protein because it still consists of the essential amino acid sequence or sequences which are responsible for the original features the protein exhibits. In other words, the fragment retains the ability to specifically bind to its target. The term “fragment” can also be understood as that the fragment has a deletion of up to 1, 2, 3, 4, 5, 6, 7, 8, 9, 10, 15, 20, 25, 30, 35, 40, 45, 50, 55, 60 amino acids at its N-terminus and/or at its C-terminus and/or internally as compared to the parental polypeptide, peptide or protein preferably at its N-terminus, at its N- and C-terminus, or at its C-terminus.

The term “antigen” as used in the context of the present invention refers to any structure recognized by molecules of the immune response, e.g. antibodies, T cell receptors (TCRs) and the like. An antigen may be foreign or toxic to the body or may be a cellular protein that is associated with a particular disease. Antigens are recognized by highly variable antigen receptors (B-cell receptor or T-cell receptor) of the adaptive immune system and may elicit a humoral or cellular immune response. Antigens that elicit such a response are also referred to as immunogen. A fraction of the proteins inside cells, irrespective of whether they are foreign or cellular, are processed into smaller peptides and presented by the major histocompatibility complex (MHC). A cellular immune response is elicited, if the small peptide fragment is bound by a T-cell receptor. Cell surface antigens can be selected from the group of cytokine receptors, integrins, cell adhesion molecules, cell type-specific cell surface antigens, tissue-specific cell surface antigens, cell surface-expressed tumor-associated antigens, tumor-antigens, cluster of differentiation antigens, or carbohydrates.

The term “single chain antibody” is interchangeably used with the term single chain variable fragment (scFv) and refers to a target specific binding domain, which usually is a polypeptide facilitating specific binding to a target. The binding of such a target-specific binding domain is considered specific to a given target if it binds with the highest affinity to the respective target and only with lower affinity, e.g. at least 10-fold lower, preferably at least 100-fold lower affinity to other targets even to targets with a related amino acid sequence. A scFv is not actually a fragment of an antibody, but instead comprises the heavy chain variable domain joined via a short linker peptide to the light chain variable domain (Huston et al. (1988) Proc. Natl. Acad. Sci. USA 85, 5879-5883). The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

The term “single chain antibody-like protein” refers to a scFv protein that has been engineered (e.g. by mutagenesis of loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin repeat proteins (for review see: Binz H. K. et al. (2005) Engineering novel binding proteins from non-immunoglobulin domains. Nat. Biotechnol. 23(10):1257-1268). Antibody-like proteins can be derived from large libraries of mutants, e.g. be panned from large phage display libraries and can be isolated in analogy to regular antibodies. Also, antibody-like binding proteins can be obtained by combinatorial mutagenesis of surface-exposed residues in globular proteins. Antibody-like proteins are sometimes referred to as “peptide aptamers”.

The term “nucleic acid” or “polynucleotide” as used in this specification comprises polymeric or oligomeric macromolecules, or large biological molecules, essential for all known forms of life. Nucleic acids, which include DNA (deoxyribonucleic acid) and RNA (ribonucleic acid), are made from monomers known as nucleotides. Most naturally occurring DNA molecules consist of two complementary biopolymer strands coiled around each other to form a double helix. The DNA strand is also known as polynucleotides consisting of nucleotides. Each nucleotide is composed of a nitrogen-containing nucleobase as well as a monosaccharide sugar called deoxyribose or ribose and a phosphate group. Naturally occurring nucleobases comprise guanine (G), adenine (A), thymine (T), uracil (U) or cytosine (C). The nucleotides are joined to one another in a chain by covalent bonds between the sugar of one nucleotide and the phosphate of the next, resulting in an alternating sugar-phosphate backbone. If the sugar is desoxyribose, the polymer is DNA. If the sugar is ribose, the polymer is RNA. Typically, a polynucleotide is formed through phosphodiester bonds between the individual nucleotide monomers. In the context of the present invention the term “nucleic acid” includes but is not limited to ribonucleic acid (RNA), deoxyribonucleic acid (DNA), and mixtures thereof such as e.g. RNA-DNA hybrids (within one strand), as well as cDNA, genomic DNA, recombinant DNA, cRNA and mRNA. A nucleic acid may consist of an entire gene, or a portion thereof, the nucleic acid may also be a miRNA, siRNA, piRNA or shRNA. MiRNAs are short ribonucleic acid (RNA) molecules, which are on average 22 nucleotides long but may be longer and which are found in all eukaryotic cells, i.e. in plants, animals, and some viruses, which functions in transcriptional and post-transcriptional regulation of gene expression. MiRNAs are post-transcriptional regulators that bind to complementary sequences on target messenger RNA transcripts (mRNAs), usually resulting in translational repression and gene silencing. Small interfering RNAs (siRNAs), sometimes known as short interfering RNA or silencing RNA, are short ribonucleic acid (RNA molecules), between 20-25 nucleotides in length. They are involved in the RNA interference (RNAi) pathway, where they interfere with the expression of specific genes. A short hairpin RNA (shRNA) or small hairpin RNA (shRNA) is an artificial RNA molecule with a tight hairpin turn that can be used to silence target gene expression via RNA interference (RNAi). Expression of shRNA in cells is typically accomplished by delivery of plasmids or through viral or bacterial vectors. PiRNAs are also short RNAs which usually comprise 26-31 nucleotides and derive their name from so-called piwi proteins they are binding to. The nucleic acid can also be an artificial nucleic acid. Artificial nucleic acids include polyamide or peptide nucleic acid (PNA), morpholino and locked nucleic acid (LNA), as well as glycol nucleic acid (GNA) and threose nucleic acid (TNA). Each of these is distinguished from naturally-occurring DNA or RNA by changes to the backbone of the molecule. The nucleic acids, can e.g. be synthesized chemically, e.g. in accordance with the phosphotriester method (see, for example, Uhlmann, E. & Peyman, A. (1990) Chemical Reviews, 90, 543-584).

The term “immune response” refers to a reaction of the immune system triggered by a reaction of an antigen and a molecule and/or cells of the adaptive immune system. Such immune responses may either be humoral immune responses or cellular immune responses. A humoral immune response is elicited by B-cell epitopes, wherein a cellular immune response is elicited by T cell epitopes.

The term “treat”, “treating”, “treatment” or “therapy” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that has previously had the disorder(s); and (e) limiting or preventing recurrence of symptoms in individuals that were previously symptomatic for the disorder(s). Accordingly, a moiety having a therapeutic effect treats the symptoms of a disease or disorder by accomplishing one or more of above named effects (a)-(e).

As used herein, “prevent”, “preventing”, “prevention”, or “prophylaxis” of a disease or disorder means preventing that such disease or disorder or side effect occurs in patients.

The term “tumor lysis syndrome” (TLS) refers to an oncometabolic emergency resulting from rapid cell death. Tumor lysis syndrome can occur as a consequence of tumor-targeted therapy or spontaneously. A group of metabolic abnormalities can occur as a complication during the treatment of cancer, where large amounts of tumor cells are killed off (lysed) at the same time by the treatment, releasing their contents into the bloodstream. This occurs most commonly after the treatment of lymphomas and leukemias. Tumor lysis syndrome is characterized by high blood potassium (hyperkalemia), high blood phosphorus (hyperphosphatemia), low blood calcium (hypocalcemia), high blood uric acid (hyperuricemia), and higher than normal levels of blood urea nitrogen and other nitrogen-containing compounds. These changes in blood electrolytes and metabolites are a result of the release of cellular contents of dying cells into the bloodstream from breakdown of cells. In TLS, the breakdown occurs after cytotoxic therapy or from cancers with high cell turnover and tumor proliferation rates. The metabolic abnormalities seen in tumor lysis syndrome can ultimately result in nausea and vomiting, but more seriously acute uric acid nephropathy, acute kidney failure, seizures, cardiac arrhythmias, and death.

### EMBODIMENTS

In the following, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As mentioned in the background section, CAR T cell therapy may lead to TLS, to a so-called cytokine storm in a worst case scenario as a side effect which is due to the specific activity of the CAR T cells. To curtail the consequences of increased cytokine release, treatment options include the use of an anti-IL-6 antibody or administration of corticosteroids, which either leads to insufficient therapy of the primary disease, e.g. cancer. Thus, it is generally desirable to prevent or at least decrease the probability of TLS during CAR T cell therapy. In the work leading to the present invention, it was surprisingly shown by the inventors that an AAV-derived modified VSP, in particular VCP carrying an epitope (LCAR) which specifically binds to its respective CAR shows transient non-availability of the CAR at the cell surface by internalisation of the CAR. Preferably the cells used for CAR therapy are T cells. Further, the inventors could show that this non-availability is time controllable. In consequence, the transient non-availability of the CAR leads to decreased or no TLS but, however, T cells do not undergo apoptosis and thus, are still available for cancer therapy.

This surprising finding may provide inter alia the following advantages over the art: (i) reduction/prevention of TLS; (ii) guarantee of target-specific T-cells in the patient due to prevention of T-cell apoptosis; (iii) can be implemented for each CAR of interest once the binding motif has been identified; (iv) no additional activation of CAR T cells by binding of VSP fusion proteins of the invention.

In a first aspect the present invention relates to a VSP, wherein:
(i) the VSP, preferably VCP is capable of, optionally together with one or more further viral coat proteins forming a capsomeric structure, a capsid, a virus-like particle (VLP), a viral vector or a virus,
(ii) the VSP, preferably VCP comprises in a region that is located on the surface of said capsomeric structure, capsid, VLP, viral vector or virus at least one ligand specifically binding to a chimeric antigen receptor (LCAR), and wherein the LCAR is a polypeptide.

The ability of a given VSP, preferably VCP to form multimeric structures, capsomeric structure, a capsid, a virus like particle (VLP), a viral vector or a virus can be assessed by several art-known processes like, e.g. electron microscopy, size exclusion chromatography or non-denaturing gel electrophoresis. The skilled person can easily assess whether a VSP, preferably VCP, e.g. a naturally occurring VSP, preferably VCP, that has been modified by the insertion of an LCAR is still capable of forming non-covalent bonds with other VSPs, preferably VCPs of the same type and/or other types of VSPs, preferably VCPs, preferably under physiological conditions. The insertion is least likely to interfere with self-assembly, if the LCAR is inserted in a region of the VSP, preferably VCP that is not an interaction surface for the VSPs, preferably VCPs. Consistently, a surface of the capsomeric structure, capsid, VLP, viral vector or virus is that part of the protein complex that after assembly of the respective structure is still available to interact with other proteins that are in solution around the complex or which are at the surface of a cell, like a CAR. Someone of the skill in structural biology can use x-ray crystallography and/or H1-NMR spectroscopy to determine the three-dimensional structure of each individual VSP, preferably VCP as well as of the capsomeric structure, capsid, VLP, viral vector or virus and determine amino acids of the VSPs, preferably VCPs forming that structure that are located at the surface. These amino acid positions are preferred sites of insertion, since they are least likely to disrupt the overall structure of the VSP, preferably VCP and/or to interfere with the self-assembly of the VSP, preferably VCP. Accordingly, the phrase “region that is located on the surface” refers to one or more adjacent amino acids that are surface-exposed as described above. It is also preferred that the region is not located in a helical and/or beta-sheet structure of the VSP, preferably VCP, since the insertion of an LCAR into such structures may disrupt the secondary structure of the VSP and, thus may interfere with self-assembly. Accordingly, it is preferred that the LCAR is inserted into a surface exposed and unstructured region of the VSP, preferably VCP, preferably a loop structure.

In an embodiment of the first aspect of the invention the VSP, preferably VCP is a virus derived coat protein which is capable of assembling together with one or more further VSP, preferably VCPs to capsomeric structures. Capsomeric structures are multimers, preferably pentamers, hexamers or and several of these subunits assemble to form a capsid, a VLP, viral vector or a virus. Without wishing to be bound by any theory the inventors believe that the surprising finding of the present invention, i.e. the reduction of side effects caused by CAR therapy, is at least in part based on the fact that the VSP, preferably VCPs of the present invention, and in particular if assembled into capsomers, capsids, VLPs, viral vectors or viruses, are binding to the respective CAR and in turn control the availability of said CAR on the T cell surface.

In another embodiment of the first aspect of the present invention it is preferred that one or more VSP, preferably VCP form capsomeric structures, a capsid, a VLP, a viral vector or a virus and that the VSP, preferably VCP comprises in a region which is located on the surface of said capsomeric structure, capsid, VLP, viral vector or virus at least one ligand which is specifically binding to CAR (LCAR) and which is a polypeptide. Preferably, this LCAR comprises or consists of 50 amino acids in length, more preferably the LCAR comprises or consists of 5-50, 6-30, 7-20, 8-15 amino acids. In a more preferred embodiment the LCAR comprises or consists of 9-15 amino acids or consists of 13 amino acids.

The present inventors have discovered that AAV2 modified to comprise an LCAR binds primarily, if not exclusively to the T-cell through the CAR expressed on the T-cell and then undergoes endocytosis. Without wishing to be bound by any theory, the inventors believe that the binding to the CAR triggers endocytosis and is independent of the normal viral entry pathways of AAV into T cells. In fact, the present inventors have detected almost exclusive binding of the modified AAV to CAR. This observation is a strong indication that it is the interaction between the LCAR present in the VSP and the CAR that is pivotal in triggering endocytosis and it is not the interaction with the surface proteins that AAVs otherwise interact for entry. On the basis of this observation, the present inventors consider it credible that VSPs of other viruses in particular VCPs can be modified to comprise a LCAR and that such modified VSPs will also bind specifically to the CAR and trigger the same endocytotic pathway that reduces the CAR concentration on the surface of the T-cell and, thus the T-cell response. Since endocytosis is mediated by CAR, the natural entry pathway of a given virus is of lesser significance and thus VSPs and correspondingly capsomeric structure, a capsid, a VLP, a viral vector or a virus comprising such VSPs can be used which naturally enter cells through endocytosis or fusion with the cell membrane. It is, however, preferred that viruses are used that enter the cell through endocytosis. AAV is a non-enveloped virus. All non-enveloped viruses infect cells through endocytosis. Accordingly, it is particularly preferred that the VSP is from a non-enveloped virus.

Any VSP, preferably VCP self-assembled to form a higher order structure will have amino acid regions that are surface exposed and, thus may comprise an LCAR. Therefore, in another embodiment of the first aspect of the invention the VSP, preferably VCP is derived from a virus selected from the group consisting of double-stranded DNA viruses, preferably Myoviridae, Siphoviridae, Podoviridae, Herpesviridae, Adenoviridae, Baculoviridae, Papillomaviridae, Polydnaviridae, Polyomaviridae, Poxviridae; single-stranded DNA viruses, preferably Anelloviridae, Inoviridae, Parvoviridae; double-stranded RNA viruses, preferably Reoviridae; single-stranded RNA viruses, preferably Coronaviridae, Picornaviridae, Caliciviridae, Togaviridae, Flaviviridae, Astroviridae, Arteriviridae, Hepeviridae; negative-sense single-stranded RNA viruses, preferably Arenaviridae, Filoviridae, Paramyxoviridae, Rhabdoviridae, Bunyaviridae, Orthomyxoviridae, Bornaviridae; single-stranded RNA reverse transcribing viruses, preferably Retroviridae; double-stranded DNA reverse transcribing viruses, preferably Caulimoviridae, Hepadnaviridae.

Preferably the VSP, preferably the VCP is derived from a virus that enters the cells by endocytosis wherein the virus is selected from the group consisting of Myoviridae, Siphoviridae, Podoviridae, Herpesviridae, Adenoviridae, Baculoviridae, Papillomaviridae, Polyomaviridae, Poxviridae; Anelloviridae, Inoviridae, Parvoviridae; Reoviridae, Coronaviridae, Picornaviridae, Caliciviridae, Togaviridae, Flaviviridae, Astroviridae, Arteriviridae, Hepeviridae, Arenaviridae, Filoviridae, Rhabdoviridae, Bunyaviridae, Orthomyxoviridae, Bornaviridae, Caulimoviridae, and Hepadnaviridae.

Preferably the VSP, preferably the VCP is derived from a non-enveloped virus wherein the virus is selected from the group consisting of Myoviridae, Siphoviridae, Podoviridae, Adenoviridae, Papillomaviridae, Polyomaviridae, Anelloviridae, Inoviridae, Parvoviridae, Reoviridae; Picornaviridae, Caliciviridae, Astroviridae, Hepeviridae, and Caulimoviridae.

Preferably the VSP, preferably the VCP is derived from a non-enveloped single-stranded DNA virus wherein the virus is selected from the group consisting of Anelloviridae, Inoviridae, and Parvoviridae, most preferably of a Parvoviridae.

Preferably, the VCP is capable of mediating attachment to host cell surface proteins facilitating endocytotic or phagocytotic uptake of the virus into the cell, e.g. Myoviridae tail fiber proteins, Siphoviridae tail fiber and tail tip proteins, Podoviridae tail fiber proteins, Herpesviridae gB, gC, gD and gH proteins, Adenoviridae capsid proteins fiber, hexon, penton, Baculoviridae major envelope glycoproteins, Papillomaviridae structural proteins L1 and L2, Polyomaviridae capsid protein VP1, Inoviridae: g3p protein, Parvoviridae capsid proteins, Reoviridae σ1 protein, Coronaviridae S and HE proteins, Picornaviridae capsid proteins VP1, VP2, VP3, Caliciviridae capsid protein VP1, Togaviridae Glycoproteins E1 and E2, Flaviviridae M and E proteins, Astroviridae capsid proteins VP25, VP27, VP34, Arteriviridae major glycoprotein GP5, membrane protein M, minor glycoproteins GP2a, GP3, GP4, small hydrophobic proteins E and ORF5a protein, Hepeviridae capsid proteins, Arenaviridae GP glycoproteins, Filoviridae GP glycoproteins, Paramyxoviridae HN, H or G glycoproteins, Rhabdoviridae G glycoproteins, Bunyaviridae Glycoproteins Gn and Gc, Orthomyxoviridae HA protein, Bornaviridae GP glycoproteins, Retroviridae SU glycoprotein, Caulimoviridae Capsid proteins, virus-associated protein (VAP), Hepadnaviridae glycoproteins S, M and L. More preferably, the VCP is from a family of the Parvoviridae, preferably from adeno-associated virus. Even more preferably, the AAV is human AAV, bovine AAV, caprine AAV, avian AAV, canine parvovirus (CPV), mouse parvovirus; minute virus of mice (MVM); parvovirus B19 (B19); parvovirus H1 (H1); human bocavirus (HBoV); feline panleukopenia virus (FPV); or goose parvovirus (GPV). In case of each of the above mentioned VCPs, the structure of the protein is well known to the skilled person. Thus, the amino acid region of each of the VCPs that is located on the surface of a capsomeric structure, a capsid, a VLP, a viral vector or a virus comprising such VCP is also known and, therefore, the skilled person can determine amino acid positions within these VCPs suitable for insertion LCARs (see, e.g. WO 2017/167988 regarding adenoviral VLPs engineered to include peptides in surface exposed parts of hexon or fibre).

Even more preferably, the VCP is from a certain AAV-serotype, preferably AAV-1, AAV-2, AAV-2-AAV-3 hybrid, AAV-3a, AAV-3b, AAV-4, AAV-5, AAV-6, AAV-6.2, AAV-7, AAV-8, AAV-9, AAV-10, AAVrh.10, AAV-11, AAV-12, AAV-13 or AAVrh32.33. More preferably, the VCP is from AAV-2, AAV-5, AAV-8, AAV-9 or a variant thereof with at least 80%, more preferably at least 85%, even more preferably at least 90% and most preferably at least 95% amino acid sequence identity and that is capable of assembling into a capsomeric structure, a capsid, a VLP, a viral vector or a virus, preferably into a VLP.

It is also preferred that the VSP, preferably VCP can be derived from two or more different viruses of the same type, e.g. two or more AAVs of different serotypes or two or more adenovirus of different serotypes, i.e. a chimeric VSP, preferably a chimeric VCP.

Such chimeric VSPs, preferably VCPs can be generated by specific exchange of corresponding parts of two or more VSPs, preferably VCPs. For example a chimeric VP1 of AAV2 and AAV3 may comprise the amino acid sequence of AAV2 from AAHP 1 to 360 and the amino acid sequence of AAV3 from AAHP 361 to 761 (see AAHP numbering as used in FIG. 12). Alternatively, chimeric VSPs of two, three, four, five or more VSPs can be generated by random methods using, e.g. DNAs digested as described in Grimm et al. (2008) J Virol. 82(12):5887-911). To use such random methods it is preferred that the amino acids of the same different strains or serotypes are homologous to each other. Preferably, the VSPs have an amino acid identity of at least 50% over the entire length of the aligned VSPs (see FIG. 12 for such an exemplary alignment in which each VCP included in the Figure has at least 50% amino acid sequence identity to each other).

In another embodiment of the first aspect of the invention it is preferred that the VCP is selected from the group consisting of VP1, VP2 or VP3. More preferably the VCP consists of VP. More preferably, the VCP comprises VP1 according to SEQ ID NO: 1 or a variant thereof with at least 80%, more preferably at least 85%, even more preferably at least 90% and most preferably at least 95% amino acid sequence identity to the amino acid of SEQ ID NO: 1 and that is capable of assembling into a capsomeric structure, a capsid, a VLP, a viral vector or a virus, preferably into a VLP.

In another embodiment of the first aspect of the invention the insertion site of one or more LCAR(s) within the AAV VCPs is described with reference to AAV-derived VP1 according to SEQ ID NO: 1. Thus, the preferred insertion sites for the one or more LCAR(s) within VP1 according to SEQ ID NO: 1 are at different positions within the different VP proteins due to the shifted open reading frame of the amino acid sequence. Preferably, the one or more LCAR(s) is inserted at one, two or more amino acid positions (insertion sites) selected from the group consisting of M1, P34, T138, A139, K161, S261, A266, N381, R447, T448, G453, R459, R471, F534, T573, Q584, N587, R588, A591, P657, A664, T713 or T716, preferably R588 of VP1 according to SEQ ID NO: 1 or a variant thereof with at least 80%, more preferably at least 85%, even more preferably at least 90% and most preferably at least 95% amino acid sequence identity to the amino acid of SEQ ID NO: 1 and that is capable of assembling into a capsomeric structure, a capsid, a VLP, a viral vector or a virus, preferably into a VLP, or at an analogous position of VP1 of a different AAV serotype or a variant thereof; preferably at amino acid position R588 and G453 or at analogous positions of VP1 of a different AAV serotype. Typically, the expression of a nucleic acid encoding a VP1 modified as outlined above leads to the generation of three different proteins, i.e. VP1, VP2 and VP3, wherein VP2 and VP3 are N-terminally truncated variants of VP1 resulting from alternative splicing (VP2) or leaky scanning (VP3), respectively. Accordingly, the resulting assembled capsomeric structure, virus etc. will comprise VP1, VP2 and VP3 all comprising the inserted LCAR, however, at different positions. This is due to the fact that, e.g. S261 of VP1 is located in VP2 at position S124 and in VP3 at position S59. Alternatively, it is preferred that the insertion site of the one or more LCAR(s) is at one, two or more insertion sites selected from the group consisting of M1, A2, K24, S124, A129, N244, R310, T311, G316, R322, R334, F397, T436, Q447, N450, R451, A454, P520, A527, T576 or T579, preferably R451 of VP2 according to SEQ ID NO: 2 or a variant thereof with at least 80%, more preferably at least 85%, even more preferably at least 90% and most preferably at least 95% amino acid sequence identity to the amino acid of SEQ ID NO: 2 and that is capable of assembling into a capsomeric structure, a capsid, a VLP, a viral vector or a virus, preferably into a VLP, or at an analogous position of VP2 of a different AAV serotype or a variant thereof; preferably at amino acid position R451 and G316 or at analogous positions of VP2 of a different AAV serotype. Alternatively, is preferred that the insertion site of the one or more LCAR(s) is at one, two or more insertion sites selected from the group consisting of S59, A64, N179, R245, T246, G251, R257, R269, F332, T371, Q382, N385, R386, A389, P455, A462, T511 or T514, preferably R386 of VP3 according to SEQ ID NO: 3 or a variant thereof with at least 80%, more preferably at least 85%, even more preferably at least 90% and most preferably at least 95% amino acid sequence identity to the amino acid of SEQ ID NO: 3 and that is capable of assembling into a capsomeric structure, a capsid, a VLP, a viral vector or a virus, preferably into a VLP, or at an analogous position of VP3 of a different AAV serotype or a variant thereof; preferably at amino acid position R386 and G251 or at analogous positions of VP3 of a different AAV serotype. In this context the term “insertion site” means that the LCAR polypeptide is inserted C-terminally of the particularly indicated amino acid residue. Thus, an insertion of the amino acid sequence LLLL at site K24 will results in the following amino acid sequence within the VCP: KLLLL. It is also possible that the insertion is accompanied with deletions of further amino acid sequences of VCPs.

Thus, in a particular embodiment the LCAR is comprised in the VP1 of another AAV serotype at an amino acid position that is analogous to one of M1, P34, T138, A139, K161, S261, A266, N381, R447, T448, G453, R459, R471, F534, T573, Q584, N587, R588, A591, P657, A664, T713 or T716 of AA2 VP1 according to SEQ ID NO: 1. As has been described above the term analogous position refers to the amino acid in the VP1 of an AAV of another serotype that alignes with one of the amino acids of VP1 Of AAV2 when aligned (see FIG. 12). Accordingly, it is preferred that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV1 according to SEQ ID NO: 19; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV3a according to SEQ ID NO: 20; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV3b according to SEQ ID NO: 21; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV4 according to SEQ ID NO: 22; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV5 according to SEQ ID NO: 23; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV6 according to SEQ ID NO: 24; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV6.2 according to SEQ ID NO: 25; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV7 according to SEQ ID NO: 26; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV8 according to SEQ ID NO: 27; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV9 according to SEQ ID NO: 28; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV10 according to SEQ ID NO: 29; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV11 according to SEQ ID NO: 30; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV12 according to SEQ ID NO: 31; that the LCAR is at a position analogous to M1, P34, T138, A139, K161, S261, A266, N381, R447, T448, G453, R459, R471, F534, T573, Q584, N587, R588, A591, P657, A664, T713 or T716, preferably analogous to R588 of VP1 of AAV13 according to SEQ ID NO: 32; that the LCAR is at a position analogous to M1, P34, T138, A139, K161, 5261, A266, N381, R447, T448, G453, R459, R471, F534, T573, Q584, N587, R588, A591, P657, A664, T713 or T716, preferably analogous to R588 of VP1 of AAV.rh.10 according to SEQ ID NO: 33; or that the LCAR is at a position analogous to M1, P34, T138, A139, K161, 5261, A266, N381, R447, T448, G453, R459, R471, F534, T573, Q584, N587, R588, A591, P657, A664, T713 or T716, preferably analogous to R588 of VP1 of AAVrh32.33 according to SEQ ID NO: 34.

In a particular embodiment the LCAR is comprised in a variant of a naturally occurring VP1 protein, preferably a variant of a VP1 protein according to SEQ ID NO: 19, 20, 21, 22, 23, 24, 25, 26, 27, 28, 29 30, 31, 32, 33, or 34. In a particular embodiment such a variant has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a VP1 protein according to SEQ ID NO: 19, 20, 21, 22, 23, 24, 25, 26, 27, 28, 29 30, 31, 32, 33, or 34. In each case the variant is capable of assembling into a capsomeric structure, a capsid, a VLP, a viral vector or a virus, preferably into a VLP.

In another embodiment of the first aspect of the invention it is preferred that one or more amino acids of VP1 C-terminal to one, two or more insertion sites of the one or more LCAR are deleted. Typically between 5 to 50, 6 to 30, 7 to 20, or 8 to 15 amino acids of VP are deleted. Preferably, the number of deleted amino acids corresponds to the number of the inserted LCAR amino acids. Additionally or alternatively, it is also preferred that one or more amino acids are substituted C-terminally or N-terminally of the insertion sites. More preferably, the one or more amino acids substituted C-terminally or N-terminally are substituted within ten amino acids of the insertion site.

In another embodiment of the first aspect of the invention the LCAR inserted into the VCP sequence is N- and/or C-terminally flanked by a linker. Examples for linkers are short polypeptide sequences, e.g. of 5, 10, 15, 20 and 25 amino acid residues length. Preferred linkers comprise alanine/glycine/serine linkers. The linkers increase the flexibility of the LCAR flanked N- and/or C-terminally by linkers and, thus increase its accessibility, e.g. to bind to CARs.

In another embodiment of the first aspect of the invention it is preferred that the LCAR inserted into the VSP, preferably VCP comprises or consists of one, two, three or more surface accessible epitopes of a TSEA. Preferably, this LCAR comprises, essentially comprises or consists of 50 or less continuous amino acids of the TSEA, more preferably the LCAR comprises, essentially comprises or consists of between 5 to 50, 6 to 30, 7 to 20, 8 to 15 amino acids. In a more preferred embodiment the LCAR comprises 9 to 15 amino acids or consists of 13 amino acids.

Preferably, the TSEA, in particular the LCAR does not comprise B cell epitopes and preferably also no T cell epitopes. This is preferred because in this embodiment the modified VSP, preferably VCP or capsomers, capsids, VLPs, viral vectors or viruses comprising such VSPs, preferably VCPs will not elicit an immune response. Thus, C- and/or N-terminally deleted variants of antigens can be used in as long as the they are still specifically bound by a CAR.

Preferably, the TSEA is selected from the group consisting of: cancer testis antigens, preferably NY-BR1, MAGE-A1, IL13Ra2, NY-ESO-1; oncofetal antigens, preferably CEA, EphA2, PSCA, L1-CAM; differentiation antigens, preferably CD19, CD20, CD2,2 CD30, CD33, CD44, CD44v6, CD70, CD123, CD138, CD171, DLL3, EGFR, EGFRvIII, EpCAM, FAP, GPC3, HER2, Mesothelin, MG7, PSMA, gp100, AlphaFR, CAIX, NKG2D-L, BCMA Igk, ROR-1, cMet, VEGFR-II; viral antigens or altered glycoproteins, preferably AC133, MUC-1, GD2, Lewis-Y. It is even more preferred that the tumor-associated antigen is NY-BR. The one or more epitopes of the TSEA are accessible on the surface of the tumor cell, i.e. can be bound by an immune cell, preferably a T cell equipped with a CAR. This is a requirement for CART cell therapy since otherwise the CAR cannot bind to the target cell. Thus, preferred LCARs comprise, essentially consist or consist of one or more epitopes of a TSEA that can be bound by a CAR in CART cell therapy. Preferably, such LCARs do not comprise T-cell and/or B-cell epitopes.

In another embodiment of the first aspect of the present invention the LCAR comprised in the VSP of the present invention is modified to comprise a Cys residue in at least one epitope interacting with the CAR. If the respective CAR that is specifically bound by the LCAR naturally comprises a Cys residues in vicinity of the Cys residue of the LCAR or is engineered to comprise such a Cys residue the two can form a covalent interaction which increases the binding affinity between the LCAR and the CAR.

In another embodiment of the first aspect of the present invention the LCAR is selected from the group consisting of a single chain antibody or a single chain antibody like-protein. To be useful in modulating CAR cell therapy, the antibody specifically binds to the CAR on the immune cell expressing the CAR. In this embodiment it is particularly preferred that the antibody binds to the part of the CAR that specifically binds to its target, e.g. the protein fold formed by the CDRs of the VH and VL in case that the CAR itself comprises a single chain antibody.

In a second aspect the invention further relates to a nucleic acid encoding the VCP of the first aspect.

In a third aspect the invention further relates to a capsomeric structure, a capsid, a VLP, a viral vector or a virus comprising at least one VSP, preferably VCP according to the first aspect. In a preferred embodiment of the third aspect of the invention a VLP comprises at least one VCP of the first aspect of the invention. The use of VLPs and capsomeric structures are preferred, since they have a relatively long biological half-life and require less frequent administration and additionally they have a better safety profile then viral vector or a viruses since they do not comprise nucleic acids and are non-infectious and non-replicating.

In another preferred embodiment the viral vector or the virus is non-infectious, i.e. the virus as a disease causing organism is not liable to be transmitted through the environment and thus, does not cause a disease anymore. Therefore, it is preferred that the viral vector or virus is a mutant that does not spread an infection or any other disease. It is further preferred that viral vector or a virus are non-replicating.

In a fourth aspect the invention relates to a pharmaceutical composition comprising the VSP, preferably VCP of the first aspect, the nucleic acid of the second aspect, the capsomeric structure, a capsid, a VLP, a viral vector or a virus of the third aspect comprising at least one VSP, preferably VCP according to the first aspect of the invention, and further comprising one or more pharmaceutically acceptable carriers, diluents, excipients, fillers, binders, lubricants, glidants, disintegrants, adsorbents, and/or preservatives.

In particular embodiments, the composition of the fourth aspect contains a therapeutically effective amount of the active ingredient, i.e. the VSP, preferably VCP or the nucleic acid of the first or second aspect of the present invention, the capsomeric structure, a capsid, a VLP, a viral vector or a virus of the third aspect comprising at least one VSP, preferably VCP according to the first aspect of the invention preferably in purified form, together with a suitable amount of carrier and/or excipient so as to provide the form for proper administration to the patient. The formulation should suit the mode of administration.

The pharmaceutical compositions can take the form of solutions, suspensions, emulsion, tablets, pills, capsules, powders, sustained-release formulations and the like. The pharmaceutical composition can be formulated as a suppository, with traditional binders and carriers such as triglycerides.

For preparing pharmaceutical compositions of the present invention, pharmaceutically acceptable carriers can be either solid or liquid. Solid form compositions include powders, tablets, pills, capsules, lozenges, cachets, suppositories, and dispersible granules. A solid excipient can be one or more substances, which may also act as diluents, flavoring agents, binders, preservatives, tablet disintegrating agents, or an encapsulating material. In powders, the excipient is preferably a finely divided solid, which is in a mixture with the finely divided inhibitor of the present invention. In tablets, the active ingredient is mixed with the carrier having the necessary binding properties in suitable proportions and compacted in the shape and size desired. Suitable excipients are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. For preparing suppositories, a low melting wax, such as a mixture of fatty acid glycerides or cocoa butter, is first melted and the active component is dispersed homogeneously therein, as by stirring. The molten homogeneous mixture is then poured into convenient sized moulds, allowed to cool, and thereby to solidify. Tablets, powders, capsules, pills, cachets, and lozenges can be used as solid dosage forms suitable for oral administration.

Liquid form compositions include solutions, suspensions, and emulsions, for example, water, saline solutions, aqueous dextrose, glycerol solutions or water/propylene glycol solutions. For parenteral injections (e.g. intravenous, intraarterial, intraosseous infusion, intramuscular, subcutaneous, intraperitoneal, intradermal, and intrathecal injections), liquid preparations can be formulated in solution in, e.g. aqueous polyethylene glycol solution. A saline solution is a preferred carrier when the pharmaceutical composition is administered intravenously.

In particular embodiments, the pharmaceutical composition is in unit dosage form. In such form the composition may be subdivided into unit doses containing appropriate quantities of the active component.

The dosage administered is adapted in such to achieve the desired reduction of unwanted side effects of CAR therapy. Since the strength of the side effects will vary between patients the dosage can be individually adapted until the desired reduction of unwanted side effects is achieved.

The unit dosage form can be a packaged composition, the package containing discrete quantities of the composition, such as packaged tablets, capsules, and powders in vials or ampoules. Also, the unit dosage form can be a capsule, an injection vial, a tablet, a cachet, or a lozenge itself, or it can be the appropriate number of any of these in packaged form.

The composition, if desired, can also contain minor amounts of wetting or emulsifying agents, or pH buffering agents.

Furthermore, such pharmaceutical composition may also comprise other pharmacologically active substance such as but not limited to adjuvants and/or additional active ingredients. Adjuvants in the context of the present invention include but are not limited to inorganic adjuvants, organic adjuvants, oil-based adjuvants, cytokines, particulate adjuvants, virosomes, bacterial adjuvants, synthetic adjuvants, or synthetic polynucleotides adjuvants.

In a fifth aspect of the invention a VSP, preferably VCP, a nucleic acid, a capsomeric structure, a capsid, a VLP, a viral vector or a virus according to the first, second or third aspect and a pharmaceutical composition according to the fourth aspect of the invention for use in medicine are provided.

In particular embodiments the use in medicine is the use in the prophylaxis, treatment or diagnosis of a disorder or disease, in particular in the prophylaxis, treatment or diagnosis of tumor lysis syndrome.

A sixth aspect of the invention relates to a VSP, preferably VCP according the first aspect, a nucleic acid according to the second aspect, a capsomeric structure, a capsid, a VLP, a viral vector or a virus according to the third aspect of the invention, or a pharmaceutical composition of the fourth aspect of the invention for use in preventing, decreasing or limiting an immune response.

It is particularly preferred that the immune response that is prevented, decreased or limited is elicited by an adoptive immune therapy. Preferred adoptive immune therapies comprise T cell therapy, preferably CART cell therapy. Preferred immune responses which are prevented, decreased or limited are tumor lysis syndrome, cytokine release syndrome, neurologic toxicity, “on target/off tumor” recognition, graft-versus-host disease (GVHD) and/or anaphylaxis. It is particularly preferred that the immune response is a cellular response. It is preferred that treating means to improve the condition of a subject in need thereof. It is preferred that that exceeding reactions of the immune system e.g. increased cytokine production can be either prevented or at least decreased. TLS is characterized by a massive tumor cell death leading to the development of metabolic derangements and target organ dysfunction and go along with a high cytokines release. Cytokines released in connection with TLS are for example interleukins, e.g. IL-6.

In a seventh aspect the present invention relates to a kit of parts comprising a capsomeric structure, a capsid, a VLP, a viral vector or a virus comprising at least one VSP according to the first aspect of the invention, wherein the viral vector or virus is non-infectious and a modified cell expressing a CAR, that is specifically bound by said capsomeric structure, said capsid, said VLP, said viral vector or said virus.

Preferably, the capsomeric structure, capsid, VLP, viral vector or virus binds to the modified cell with an affinity of less than 10 μM.

Preferably, the kit of parts further comprises an instruction leaflet specifying the use of the capsomeric structure, capsid, VLP, viral vector or virus in preventing, decreasing or limiting an immune response, preferably elicited by an adoptive immune therapy.

### EXAMPLES

### Example 1: Production and Purification of Wt AAV and NY-BR1-LCAR AAV

In order to generate the NY-BR1 AAV capsid insertion mutants used in the present study, the LCAR was inserted into the threefold spike region of AAV as previously described (Michelfelder et al. (2011) PLoS ONE 6(8): e23101.). Specifically, oligonucleotides encoding the amino acid sequence LKNEQTLRADQMF were inserted at VP1 position 588 into the AAV2 helper plasmid pMT-187-XX2 (the resulting nucleic acid encoded a modified VP1 with an amino acid sequence as indicated in SEQ ID NO: 4), position T578 in AAV5 helper plasmid pMT-rep2cap5-SfiI578 (the resulting nucleic acid encoded a modified VP1 with an amino acid sequence as indicated in SEQ ID NO: 5), position N590 in AAV8 helper plasmid p5E18-VD2/8-Sfi590 (the resulting nucleic acid encoded a modified VP1 with an amino acid sequence as indicated in SEQ ID NO: 6) and position A589 in AAV9 helper plasmid p5E18-VD2/9-Sfi589 (the resulting nucleic acid encoded a modified VP1 with an amino acid sequence as indicated in SEQ ID NO: 7). The resulting plasmid construct was used instead of the wt AAV2 helper plasmid for NY-BR1 AAV production. Wt AAV2 and the NY-BR1 AAV capsid insertion mutants were produced in HEK293T cells by the adenovirus-free production method. Briefly, cells were triple-transfected using PEI and 44 g DNA per 15 cm plate in equimolar ratios of an AAV helper plasmid encoding rep and cap proteins, an AAV vector plasmid encoding GFP and an adenoviral helper construct. At 72 h post transfection, cells were lysed by repeated freeze-thaw cycles, treated with benzonase and further purified by iodixanol step gradient centrifugation. After centrifugation for 3 h at 55000 rpm and 4° C., the 40% phase containing the AAV particles was harvested. Genomic particle titers were determined by real-time LightCycler PCR against plasmid standards using transgene-specific primers. Wt AAV2 and NY-BR1 AAV2 capsid titers were analysed by ELISA using anti-capsid antibody A20 according to the manufacturer's instruction (Progen GmbH).

### Example 2: Detection of NY-BR1-LCAR AAV by ELISA

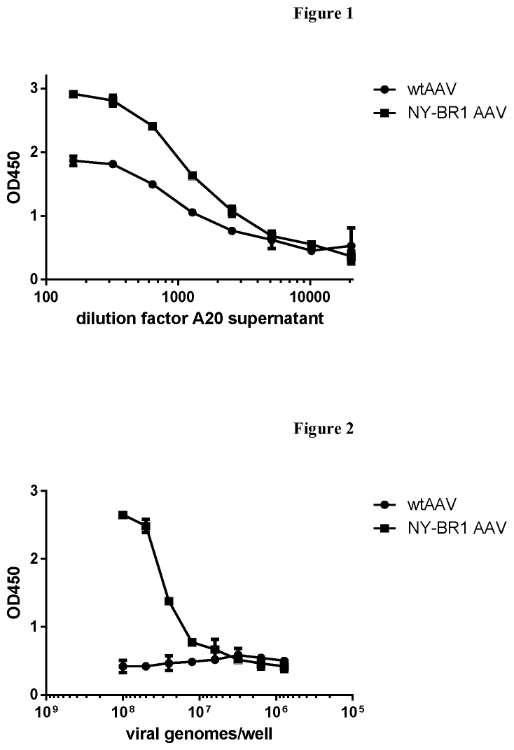

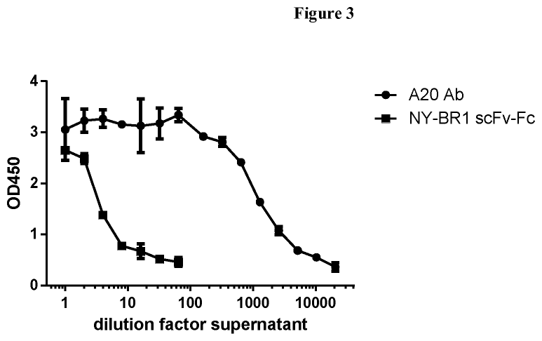

Purified NY-BR1 LCAR AAV particles were coated onto Costar® 96 well assay plate (half well) according to their determined genomic titer. Coating was performed in 50 μl of NaHCO₃ buffer/well at 4° C. overnight. Starting concentration was at 1×10⁹ viral genomes (VG)/ml, followed by 2fold serial dilution of particles in 6 steps. After incubation the plate was washed for six times with PBS-T (1×PBS+0.05% Tween20) using 150 μl/well. Blocking of the plate was conducted by incubation on a rocking device with 150 μl/well of blocking buffer (3% BSA, 5% sucrose in PBS-T) for 1 h at room temperature. Primary mouse antibody (NY-BR-1 No. 2—ThermoFisher order no.: MA5-12645) or soluble LCAR (Morab2scFv—in house production) was added to the respective wells at a final concentration of 1 g/ml in 30 μl PBS-T, followed by 1 h incubation at room temperature on a shaker. Thereafter washing with PBS-T (150 μl/well) was performed for 3 times. Secondary antibody was added with respect to the primary antibody, for the NY-BR-1 No. 2 an anti-mouse-IgG-HRP conjugate was used, for the soluble LCAR we applied an anti-human-IgG-HRP conjugate (both were purchased from Santa Cruz Biotechnology, final concentration: 1 μg/ml in 50 μl/well). Antibodies incubated for 1 h at room temperature on a shaker, followed by 3× washing with PBS-T. Detection was performed by adding TMB substrate (100 μl/well) for 15 minutes at room temperature, followed by adding of stop solution (1M H₂SO₄ 50 μl/well). The read out of OD450 was carried out on the Epoch multi plate reader (BioTek Instruments). As an internal control, a standard anti-AAV A20 ELISA was performed on the same plate following the manufacturer's instructions of the A20 ELISA Kit (Progen GmbH). Results are shown in FIG. 1-3.

### Example 3: Downregulation of NY-BR1 CAR by NY-BR1-LCAR AAV in a Cell Line

To establish a NY-BR1-CAR cell line, 1×10⁵ Jurkat cells were transduced with a lentiviral vector encoding the NY-BR1-CAR gene expression cassette and the puromycin resistance gene. Therefor Jurkat cells were seeded in 1 ml of DMEM medium (containing 10% FCS) in one well of a 24 well cell culture plate and purified lentiviral particles were added at a multiplicity of infection (MOI) of 10. Incubation for 24 h followed under standard conditions (5% CO₂; 37° C., humidified atmosphere). After that cells were washed by centrifugation (300 g; 4 min) and fresh DMEM medium was added. 3 days after transduction selection of CAR⁺-cells was carried out. Jurkat cells were washed and filled up with 20 ml of DMEM medium containing puromycin (final concentration: 2 g/ml) and 200 μl of the cells were seeded in each well of a 96 well cell culture suspension plate. Resistant clones were grown out approximately 2 or 3 weeks after seeding.

The resulting clones were further subcultivated under puromycin conditions and analysed for the expression of CAR by FACS. This was carried out with 5×10⁴ cells per analysis in which cells were incubated with an APC-conjugated anti-human-IgG1-Fc-fragment specific antibody (1 g/ml in 100 μl; Jackson Immuno Research) in FACS buffer (PBS; 1% FCS; 2 mM EDTA) for 30 min at 4° C. After washing by centrifugation, 400 μl PBS were added to the FACS tubes, and cells were counterstained for life cells with DAPI (3 μM final concentration). Detection of CAR⁺ cells was done on a FACS CantoII® device (BD Biosciences) using the FACSDiva® software. CAR expression was observed in 95-99% of cells of a respective clone.

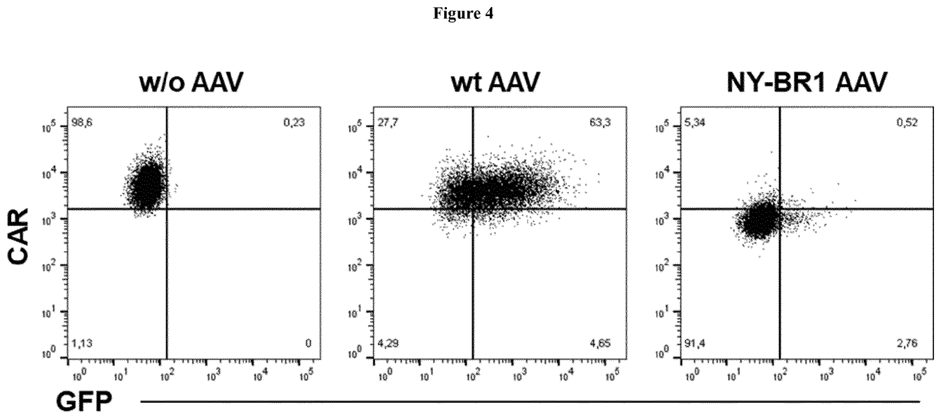

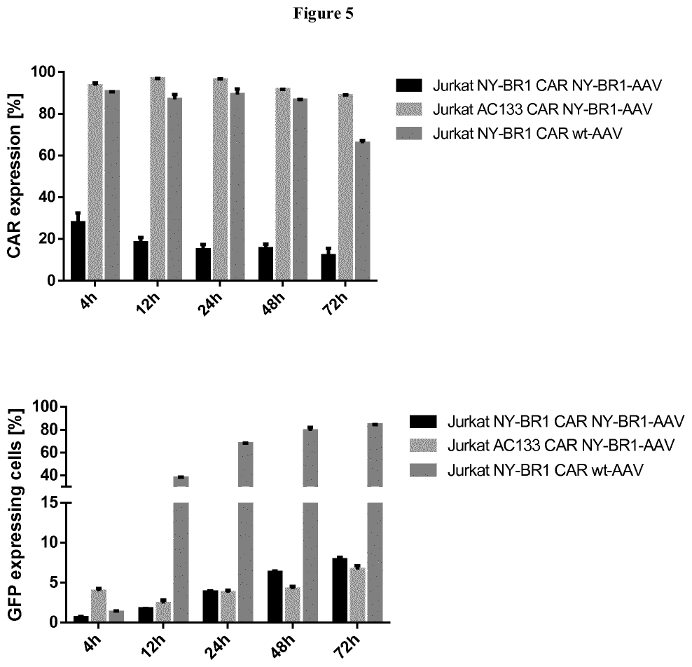

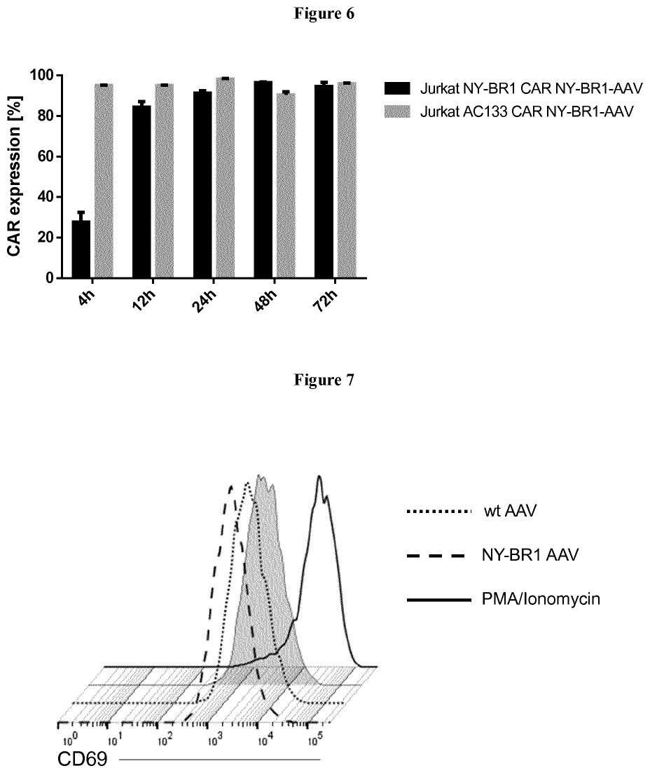

To determine the level of CAR downregulation by LCAR-AAV, CAR⁺-Jurkat cells were incubated with NY-BR1-LCAR-AAV purified as described in Example 1. 1×10⁵ CAR⁺-Jurkat cells were seeded in RPMI medium in one well of a 24 well plate. Immediately after, 5×10⁸ NY-BR-LCAR-AAV particles per well were added to the cells reflecting a MOI of 5000. Cells were then cultivated under standard conditions for 24 h. After that, FACS analysis was carried out as described above. The level of CAR downregulation was described as the change of the mean fluorescence intensity (MFI) of CAR expression in NY-BR1-LCAR-AAV incubated Jurkat cells compared to incubation with wildtype (wt) AAV (FIGS. 4-6).

### Example 4: Measurement of CAR Activation after LCAR Incubation in a Cell Line

In order to measure the level of activation in NY-BR1-CAR⁺ Jurkat cells caused by NY-BR1-LCAR-AAV particles, expression of the marker molecule CD69 was determined. CAR⁺-Jurkat cells were incubated with NY-BR1-LCAR-AAV particles and wt AAV particles under the same conditions as described in Example 3 (MOI 5000, 24 h, 37° C.). CAR⁺-Jurkat cells incubated without any AAV particles served as baseline control. Over activation as positive control was induced by PMA (20 ng/ml) and lonomycin (1 g/ml). Immediately after cultivation cells were transferred to FACS tubes and washed twice with FACS buffer followed by incubation with an anti-CD69-PECy7 antibody conjugate (Clone: FN50, BioLegend) at a concentration of 1 μg/ml in 100 μl FACS buffer on ice for 30 min. Live cell staining and FACS analysis was performed as described in Example 3. The level of activation was determined as the increase of CD69-MFI above the baseline control (FIG. 7).

### Example 5: Downregulation and CAR Activation after LCAR Incubation in Primary CAR T Cells

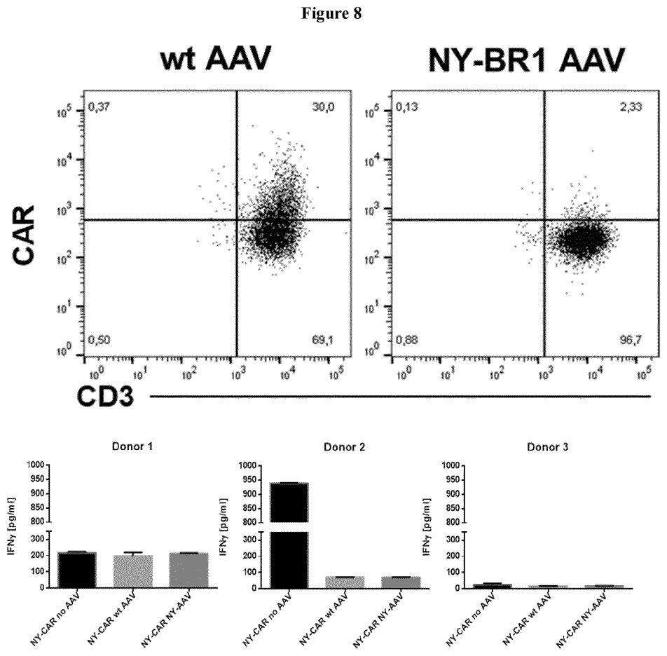

To obtain primary T cells a blood sample of around 50 ml was taken from a healthy donor in EDTA collection tubes. The fresh blood was thoroughly pipetted onto a layer of 10 ml Ficoll (Ficoll Paque™; GE Healthcare; density: 1.077) in a 50 ml Falcon™ tube and centrifugated at 750 g for 30 min without brake. After that the formed ring of lymphocytes was taken off and washed twice in 20 ml PBS (centrifugation with brake at 300 g for 4 min). Cell number was determined by counting in a Neubauer-Chamber and isolation of CD3⁺ T cells was carried out using the human Pan T cell Isolation Kit II obtained from Miltenyi Biotec and according to the manufacturer's instructions. Number of T cells was determined after sorting and 1×10⁶ cells were seeded in 1 ml of activation medium at 1 cm² for 24 h at 37° C. Activation medium consisted of XVIVO20 (Lonza) and 100 ng/ml of an anti-CD3 antibody (Clone: OKT3; Janssen-Cilag) supplemented with 300 U/ml Interleukin-2 (ProLeukin®; Novartis). After activation, T cells were washed three times in PBS and further incubated in cultivation medium (XVIVO20; 300 U/ml IL-2). 48 h after isolation of T cells lentiviral CAR transduction was carried out using the spinoculation method. Therefore cells were seeded on a Retronectin® coated 24 well plate (16 μg/ml Retronectin®; 1×10⁶ CD3⁺ cells/ml/cm²) and lentiviral particles encoding the CAR gene expression cassette were added at an MOI of 10. The plate was centrifuged for 1.5 h at 2000 g and 32° C. followed by incubation at 37° C. for 24 h. Thereafter medium containing viral particles was removed and replaced with normal cultivation medium. 72 h after initial cell sorting the rate of CAR expressing CD3+ cells was determined by FACS as described in Example 3 with the exception that here anti-CD3-APC and anti-human-IgG1-Fc-fragment-PE (each at 1 g/ml) antibody conjugates were used as detectors. To determine the functionality of NY-BR1-LCAR-AAV particles on primary CD3⁺ CAR⁺ cells the above generated cells were incubated with their respective AAV particles as described in Example 4. Measurement of CAR downregulation was carried out as in Example 3 and the level of downregulation was compared to incubation with wt AAV particles (FIG. 8). Supernatant of co-incubated T cells from this experiment was taken off the culture and stored for analysis of activation level also shown in FIG. 8. In this case the activation was determined as the amount of Interferon gamma in the culture supernatant and was detected by the BD OptEIA™ Human IFNγ ELISA set (BD Biosciences) according to the manufacturer's instructions.

### Example 6: CAR Downregulation Following Transduction with Different LCAR-AAV Serotypes

Wt AAV2 and AAV serotypes 2, 5, 8 and 9 displaying NY-BR1 LCAR at pos 588 in AAV2 and analogous positions in AAV8, AAV9 and AAV5 were produced and purified as described in Example 1. 1×10⁵ CAR⁺-Jurkat cells per well were seeded in RPMI medium in a 24 well plate. 5×10⁸ NY-BR1-LCAR-AAV genomic particles per well were added to the cells reflecting a MOI of 5000. Cells were then cultivated under standard conditions for 24 h. CAR expression was detected by FACS using an anti-human IgG-APC antibody as described above. FIG. 9 shows the level of CAR downregulation as the change of the mean fluorescence intensity (MFI) of CAR expression in NY-BR-LCAR-AAV or wtAAV2 incubated Jurkat cells compared to untreated samples.

### Example 7: CAR Binding Assay to Assess NY-BR1 LCAR Length Variation

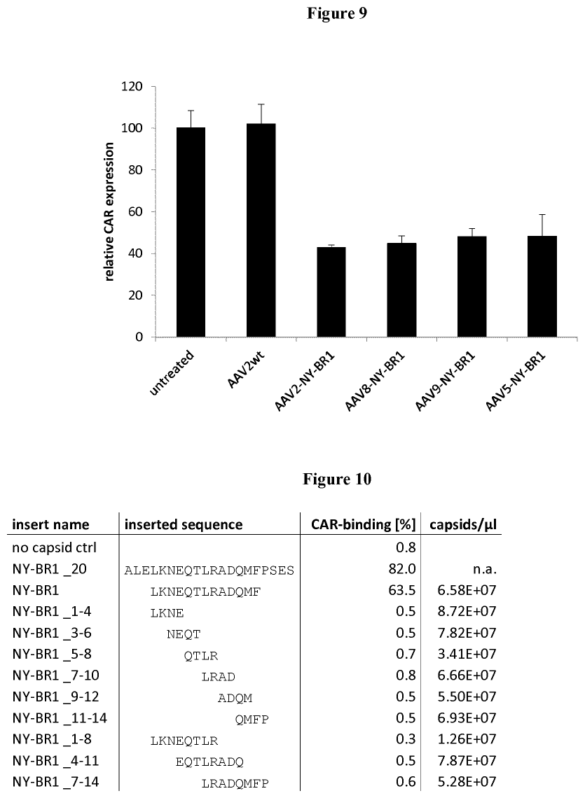

NY-BR1 LCAR sequence LKNEQTLRADQMF was split into overlapping 4mers and 8mers and elongated by the C- and N-terminal amino acids to a 20mer to vary the length of the displayed LCAR (see FIG. 10). Oligonucleotides encoding the respective amino acid sequences were inserted at VP1 position 588 into the AAV2 helper plasmid pMT-187-XX2. Crude AAV lysates were produced in HEK293T cells by the adenovirus-free production method. Briefly, cells were triple-transfected using PEI and 2.6 g DNA per well of a 6 well plate in equimolar ratios of an AAV helper plasmid encoding rep and cap proteins, an AAV vector plasmid encoding GFP and an adenoviral helper construct. At 72 h post transfection, cells were lysed by repeated freeze-thaw cycles in 100 μl PBS per well and spun down at full speed. The supernatant (i.e. crude AAV lysate) containing the AAV particles was carefully taken off. Streptavidin-coated Dynabeads (Invitrogen) were coupled with A20-biotin conjugate according to the manufacturer's instructions followed by addition of equal amounts of crude AAV lysates displaying various lengths of NY-BR1 LCAR. To quantify binding of LCAR AAVs to the soluble CAR, NY-BR1 specific scFv-Fc fusion protein was added at 0.5 g/ml. After incubation with secondary antibody anti-hu-IgG-PE, PE-positive Dynabead-AAV-Antibody complexes were detected by FACS analysis In FIG. 10, the percentage of PE-positive complexes is defined as CAR-binding [%].

### Example 8: Sandwich-ELISA of Intact AAV Particles in Crude Lysates

AAV2-specific A20 mouse hybridoma supernatant was coated onto Costar® 96 well assay plate (half well). After incubation the plate was washed three times with PBS-T (1×PBS+0.05% Tween20) using 150 μl/well. Blocking of the plate was conducted by incubation on a rocking device with 150 μl/well of blocking buffer (3% BSA, 5% sucrose in PBS-T) for 1 h at room temperature. After washing and blocking, AAV crude lysates or wt AAV2 standard were added in 2-fold serial dilutions and incubated for 1 h at room temperature. After washing as above, biotin-conjugated A20 was added at 0.6 g/ml in blocking buffer. Thereafter, washing was performed as above, followed by incubation with Streptavidin-HRP conjugate for 1 h at room temperature, followed by 3× washing with PBS-T. Detection was performed by adding TMB substrate (100 μl/well) for 15 minutes at room temperature, followed by addition of stop solution (1M H₂SO₄ 50 μl/well). The read out of OD450 was carried out on the Epoch multi plate reader (BioTek Instruments). Capsid titers were calculated by linear regression and are depicted in FIG. 10.

### Example 9: Specific Inhibition of CAR-Mediated Killing from 10-20 h Post Addition of CAR T Cells

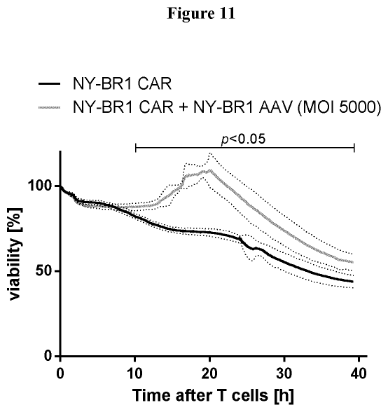

Primary breast cancer cells were obtained from ascites fluid, maintained in culture for 7 days and analyzed for expression of NY-BR1 by FACS (further named target cells). Primary T cells from a healthy donor were isolated and transduced with a lentivirus containing the expression cassette of NY-BR1 CAR as described in Example 5 (further named effector cells). In order to analyze the lytic capacity of NY-BR1 specific CAR T cells and the effect of NY-BR-LCAR AAV on them, target cells were seeded on an E-Plate 96 (20000 cells/well, triplicates per condition). The E-Plate was placed into a xCelligence real time impedance measurement device (ACEA Biosciences Inc.) and read outs were set at every 5 minutes. After 12 hours of cultivation in the device, the number of target cells had doubled and effector cells were added in an effector to target ratio of 1:1 (40000 CAR-positive T cells/well in triplicates). Further on, the co-culture was divided into two groups, one remained unchanged and the other was supplemented with NY-BR1-LCAR AAV particles at a MOI of 5000 genomic particles per CAR T cell. Impedance measurement continued for another 40 hours and was recorded by the RTCA software as cell index. At the end of the experiment, cell indices were normalized to the time point of effector cell addition and specific viability of target cells was calculated as the percentage of the normalized cell index of an untreated control. Mean viability of target cells facing CAR effector cells with or without NY-BR1-LCAR AAVs was plotted over time as shown in FIG. 11.

### Example 10: Wt AAV and NY-BR1-LCAR AAV Binding to Jurkat Cells with and without NY-BR1 CAR in the Presence or Absence of Heparin

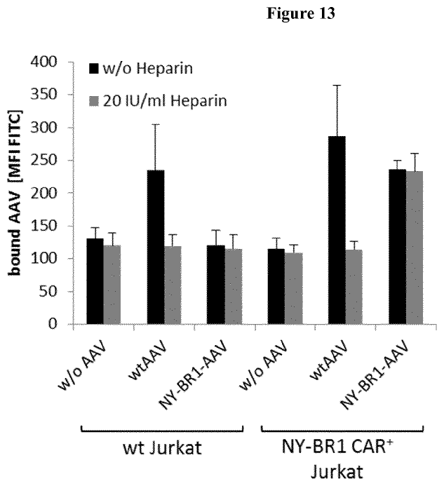

2E9 capsids of wt AAV2 or NY-BR-LCAR AAV2 were pre-incubated with 20 IU/ml heparin in 250 μl for 30 min at room temperature. 2E5 wt Jurkat cells or NY-BR1 CAR⁺ Jurkat cells were seeded in 250 μlice-cold RPMI medium per well of a 24 well plate. AAVs with or without heparin were added and the mixture was incubated on ice for 1 h. Then, cells were washed with ice-cold PBS and resuspended in 10 μl ice-cold PBS. A20-biotin conjugate (Progen) was added at 0.6 g/ml and incubated for 1 h on ice. Then, cells were washed again with ice-cold PBS and resuspended in 100 μl ice-cold PBS. Streptavidin-Alexa488 conjugate was added and incubated on ice for 30 min. Thereafter, cells were washed again with ice-cold PBS, dead cells were stained with DAPI and bound AAV was quantified by FACS analysis of live cells (FIG. 13).

1. A viral structural protein (VSP), wherein:
(i) the VSP, optionally together with one or more further VSP, is capable of forming a capsomeric structure, a capsid, a virus like particle (VLP), a viral vector or a virus; and
(ii) the VSP comprises in a region that is located on the surface of said capsomeric structure, capsid, VLP, viral vector or virus: at least one ligand specifically binding to a chimeric antigen receptor (LCAR), and wherein the LCAR is a polypeptide.

2. The VSP of claim 1, wherein the VSP is of a virus selected from the group consisting of
(i) double-stranded DNA virus,
preferably Myoviridae, Siphoviridae, Podoviridae, Herpesviridae, Adenoviridae, Baculoviridae, Papillomaviridae, Polydnaviridae, Polyomaviridae, Poxviridae;
(ii) single-stranded DNA virus,
preferably Anelloviridae, Inoviridae, Parvoviridae;
(iii) double-stranded RNA virus,
preferably Reoviridae;
(iv) single-stranded RNA virus,
preferably Coronaviridae, Picornaviridae, Caliciviridae, Togaviridae, Flaviviridae, Astroviridae, Arteriviridae, Hepeviridae;
(v) negative-sense single-stranded RNA virus,
preferably Arenaviridae, Filoviridae, Paramyxoviridae, Rhabdoviridae, Bunyaviridae, Orthomyxoviridae, Bornaviridae;
(vi) single-stranded RNA reverse transcribing virus,
preferably Retroviridae; and
(vii) double-stranded DNA reverse transcribing virus,
preferably Caulimoviridae, Hepadnaviridae.

3. The VSP according to claim 1, wherein the VSP is a viral coat protein (VCP) of a virus of the family of the Parvoviridae, preferably the VSP is from adeno-associated virus (AAV), preferably human AAV, bovine AAV (b-AAV), canine AAV (c-AAV), caprine AAV, or avian AAV (AAAV); canine parvovirus (CPV); mouse parvovirus; minute virus of mice (MVM); parvovirus B19 (B19); parvovirus HI (HI); human bocavirus (HBoV); feline panleukopenia virus (FPV); or goose parvovirus (GPV).

4. The VSP according to claim 3 wherein the AAV is AAV-1, AAV-2, AAV-2-AAV-3 hybrid, AAV-3a, AAV-3b, AAV-4, AAV-5, AAV-6, AAV-6.2, AAV-7, AAV-8, AAV-9, AAV-10, AAVrh. 10, AAV-11, AAV-12, AAV-13 or AAVrh32.33 or chimeras thereof.

5. The VSP according to any of claim 3s, wherein the VCP is selected from the group consisting of VP1, VP2 and VP3.

6. The VSP according to claim 5, wherein one or more LCARs is/are inserted:
(i) at one, two or more amino acid positions (insertion sites) selected from the group consisting of Ml, P34, T138, A139, K161, S261, A266, N381, R447, T448, G453, R459, R471, F534, T573, Q584, N587, R588, A591, P657, A664, T713 or T716, preferably R588 of VP1 according to SEQ ID NO: 1 or a variant thereof having at least 90% sequence identity to SEQ ID NO: 1, or at an analogous position of VP1 of a different AAV serotype or a variant thereof; preferably at amino acid position R588 and G453 or at analogous positions of VP1 of a different AAV serotype, or
(ii) at one, two or more insertion sites selected from the group consisting of Ml, A2, K24, S124, A129, N244, R310, T311, G316, R322, R334, F397, T436, Q447, N450, R451, A454, P520, A527, T576 or T579, preferably R451 of VP2 according to SEQ ID NO: 2 or a variant thereof having at least 90% sequence identity to SEQ ID NO: 2, or at an analogous position of VP2 of a different AAV serotype or a variant thereof, preferably at amino acid position R451 and G316 or at analogous positions of VP2 of a different AAV serotype, or
(iii) at one, two or more insertion sites selected from the group consisting of S59, A64, N179, R245, T246, G251, R257, R269, F332, T371, Q382, N385, R386, A389, P455, A462, T511 or T514, preferably R386 of VP3 according to SEQ ID NO: 3 or a variant thereof having at least 90% sequence identity to SEQ ID NO: 3, or at an analogous position of VP 3 of a different AAV serotype or a variant thereof; preferably at amino acid position R386 and G251 or at analogous positions of VP3 of a different AAV serotype
and/or
wherein preferably the LCAR is flanked C-terminally and/or N-terminally with a linker sequence.

7. The VSP according to claim 6, wherein:
(i) one or more amino acids of the VPl C- or N-terminal of the one, two or more amino acid insertion sites are deleted, and/or
(ii) one or more amino acids are substituted C-terminally or N-terminally of the insertion sites, preferably within ten amino acids of the insertion sites.

8. The VSP according to claim 1, wherein:
(i) LCAR comprises or consists of at least one epitope of a tumor surface exposed antigen (TSEA) and/or
(ii) LCAR is selected from the group consisting of a single chain antibody or a single chain antibody like-protein, specifically binding to the extracellular part of a CAR.

9. The VSP according to claim 8, wherein the TSEA is selected from the group consisting of cancer testis antigens, preferably NY-BR1, MAGE-A1, IL13Ra2, or NY-ESO-1; oncofetal antigens, preferably CEA, EpbA2, PSCA, or L1-CAM; differentiation antigens, preferably CD19, CD20, CD2,2 CD30, CD33, CD44, CD44v6, CD70, CD123, CD138, CD171, DLL3, EGFR, preferably EGFRvIII, EpCAM, FAP, GPC3, HER2, Mesothelin, MG7, PSMA, gplOO, AlphaFR, CAIX, NKG2D-L, BCMA Igk, ROR-1, cMet, or VEGFR-II; viral antigens or altered glycoproteins, preferably AC133, MUC-1, GD2, or Lewis-Y.

10. The VSP according to claim 1, wherein the LCAR has a length of 6 to 50 amino acids.

11. A nucleic acid encoding a VSP according to claim 1.

12. A capsomeric structure, a capsid, a VLP, a viral vector or a virus comprising at least one VSP according to claim 1, wherein the viral vector or virus is non-infectious.

13. (canceled)

14. A method of limiting an immune response in a subject comprising administering to the subject the capsomeric structure, the capsid, the VLP, the viral vector or the virus comprising at least one VSP according to claim 12.

15. The method of claim 14, wherein the immune response is a cellular immune response.

16. A kit of parts comprising a capsomeric structure, a capsid, a VLP, a viral vector or a virus comprising at least one VSP according to claim 1, wherein the viral vector or virus is non-infectious and a modified cell expressing a CAR, that is specifically bound by said capsomeric structure, said capsid, said VLP, said viral vector or said virus.

17. The kit of claim 16, wherein the capsomeric structure, capsid, VLP, viral vector or virus binds to the modified cell with an affinity of less than 10 μM.

18. The kit of claim 16 further comprising an instruction leaflet specifying the use of the capsomeric structure, capsid, VLP, viral vector or virus in preventing, decreasing or limiting an immune response.

19. The method of claim 14, wherein the immune response is due to the subject having received an adoptive immune therapy, wherein the therapy is a CAR cell therapy.

20. The kit of claim 18, wherein the immune response is due to a subject having received an adoptive immune therapy


SEQUENCE LISTING (34 sequences)

SEQ ID NO: 1 - protein, 735 aa, adeno-associated virus 2 [sequence omitted]

SEQ ID NO: 2 - protein, 598 aa, adeno-associated virus 2
  VP2 (1)..(589): Viral coat portein 2 of AAV serotype 2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 533 aa, adeno-associated virus 2
  VP3 (1)..(533): Viral coat protein 3 of AAV serotype 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 750 aa, Artificial Sequence
  AAV2 VP1 protein with an inserted human NY-BR antigen
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGLKNEQTLRADQMFAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNRGLTVDTNGVYSEPRPIGTRYLTRNL

SEQ ID NO: 5 - protein, 739 aa, Artificial Sequence
  AAV5 VP1 protein with an inserted human NY-BR antigen
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSGLTGLKNEQTLRADQMFAAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 753 aa, Artificial Sequence
  AAV8 VP1 protein with an inserted human NY-BR antigen
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQRGLKNEQTLRADQMFAQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 7 - protein, 751 aa, Artificial Sequence
  AAV9 VP1 protein with an inserted human NY-BR antigen
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGQAGLKNEQTLRADQMFAQAATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 20 aa, Homo sapiens
ALELKNEQTLRADQMFPSES

SEQ ID NO: 9 - protein, 13 aa, Homo sapiens
LKNEQTLRADQMF

SEQ ID NO: 10 - protein, 4 aa, Homo sapiens
LKNE

SEQ ID NO: 11 - protein, 4 aa, Homo sapiens
NEQT

SEQ ID NO: 12 - protein, 4 aa, Homo sapiens
QTLR

SEQ ID NO: 13 - protein, 4 aa, Homo sapiens
LRAD

SEQ ID NO: 14 - protein, 4 aa, Homo sapiens
ADQM

SEQ ID NO: 15 - protein, 4 aa, Homo sapiens
QMFP

SEQ ID NO: 16 - protein, 8 aa, Homo sapiens
LKNEQTLR

SEQ ID NO: 17 - protein, 8 aa, Homo sapiens
EQTLRADQ

SEQ ID NO: 18 - protein, 8 aa, Homo sapiens
LRADQMFP

SEQ ID NO: 19 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 20 - protein, 736 aa, adeno-associated virus 3a
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 21 - protein, 736 aa, adeno-associated virus 3b
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 22 - protein, 734 aa, adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 23 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 24 - protein, 736 aa, adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 25 - protein, 736 aa, adeno-associated virus 6.2
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 26 - protein, 737 aa, adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 27 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 738 aa, adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 30 - protein, 733 aa, adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 31 - protein, 742 aa, adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 32 - protein, 733 aa, adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 33 - protein, 738 aa, adeno-associated virus rh10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 34 - protein, 733 aa, adeno-associated virus rh32.33
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL